(19) United States
(12) Patent Application Publication
(10) Pub. No.: US 2014/0113009 A1
(43) Pub. Date: Apr. 24, 2014

(54) CONTROLLED APPLICATION OF CROSS-LINKING AGENT

(71) Applicant: Avedro, Inc., Waltham, MA (US)

(72) Inventors: David Muller, Boston, MA (US); Stephen M. Blinn, Amherst, NH (US); Robert Broderick, Framingham, MA (US)

(73) Assignee: Avedro, Inc., Waltham, MA (US)

(21) Appl. No.: 14/062,467

(22) Filed: Oct. 24, 2013

Related U.S. Application Data
(60) Provisional application No. 61/718,101, filed on Oct. 24, 2012.

(51) Int. Cl. A61K 47/22; A61K 33/00; A61K 41/00; A61K 31/14

(52) U.S. Cl. CPC A61K 47/22; A61K 31/14; A61K 33/00; A61K 41/00
USPC 424/717; 514/643

(57) ABSTRACT

According to aspects of the present disclosure, a kit for transepithelial delivery of a cross-linking agent to a cornea includes a first vessel containing an initial formulation that includes a cross-linking agent and a first active ingredient configured to open corneal epithelial tight junctions when applied to a cornea and one or more second vessels each containing a respective secondary formulation that includes the cross-linking agent for application to the cornea after the initial formulation is applied to the cornea.

### CROSS REFERENCE TO RELATED APPLICATIONS

This application claims priority to U.S. Provisional Application No. 61/718,101, filed Oct. 24, 2012, the contents of which are herein incorporated by reference in their entirety.

### BACKGROUND

1. Field of the Invention

The disclosure pertains to systems and methods for stabilizing corneal tissue, and more particularly, systems and methods for conveying a cross-linking agent to regions of the cornea where the cross-linking agent is activated by an initiating element.

2. Description of Related Art

A variety of eye disorders, such as myopia, keratoconus, and hyperopia, involve abnormal shaping of the cornea. Laser-assisted in-situ keratomileusis (LASIK) is one of a number of corrective procedures that reshape the cornea so that light traveling through the cornea is properly focused onto the retina located in the back of the eye. During LASIK eye surgery, an instrument called a microkeratome is used to cut a thin flap in the cornea. The cornea is then peeled back and the underlying cornea tissue ablated to the desired shape with an excimer laser. After the desired reshaping of the cornea is achieved, the cornea flap is put back in place and the surgery is complete.

The success of procedures, such as LASIK, in addressing eye disorders depends on the stability of the changes in the corneal structure after the procedures have been applied.

### BRIEF SUMMARY

Systems and approaches for stabilizing a three-dimensional structure of corneal tissue through controlled activation of cross-linking in corneal tissue are disclosed herein. Aspects of the present disclosure provide approaches for controlling the distribution of cross-linking agent within corneal tissue in three dimensions. For example, the cross-linking agent and/or the initiating element may be applied in a series of timed and controlled steps to allow distinct applications of cross-linking agent to incrementally diffuse through the corneal tissue. One or more of the applications of cross-linking agent can be applied with a concentration distinct from other applications. In addition, the activation of the cross-linking agent, once distributed, can be carried out by an initiating element applied in a pattern and with a characteristic focal depth to thereby regulate the activation of cross-linking in the corneal tissue in three dimensions. Moreover, the delivery and activation of the cross-linking agent at depths in the cornea depend on the concentration(s) of the cross-linking agent when applied and the power(s) of the initiating element.

In addition to controlling the distribution of cross-linking agent within the corneal tissue by allowing the cross-linking agent to diffuse within the cornea, additional agents can be applied to the eye to influence the diffusion of the cross-linking agent along the depth of the corneal tissue. In some embodiments, a second (neutral) compound may be applied after one or more of the concentrations of the cross-linking agent have been applied. The second compound applies a pressure to the cross-linking agent and promotes diffusion of the cross-linking agent to depths of the cornea. For example, the neutral compound may be applied at a time when diffusion of the cross-linking agent has slowed and needs to be encouraged by the neutral compound. Concentrations of cross-linking agent at varying depths of the cornea may also be controlled by applying a reverse osmotic substance, e.g., a fluid, gel, etc., to the surface of the cornea to draw cross-linking agent from the corneal tissue, particularly from regions near the surface of the cornea. In some embodiments, iontophoresis is employed to urge cross-linking agent past the epithelium and through the corneal tissue via electromotive forces applied to charged cross-linking agent.

Furthermore, aspects of the present disclosure provide systems and approaches for influencing the permeability of corneal tissue to a cross-linking agent by applying heat energy or ultrasound energy to the corneal tissue. Aspects of the present disclosure may include a delivery system that accurately and precisely delivers the initiating element to corneal fibrils according to a selected pattern. In embodiments where the initiating element is UV light, the delivery system may deliver the UV light in the form of a laser.

According to still another aspect of the present disclosure a method is disclosed for activating cross-linking in at least one eye component of an eye. The method includes conveying the cross-linking agent to a surface of the at least one eye component. The method also includes allowing a time to pass to allow the cross-linking agent to diffuse within the at least one eye component. The method includes applying a second cross-linking agent which also includes allowing a time to pass to allow the cross-linking agent to diffuse within the at least one eye component. The method also includes applying a reverse osmotic fluid to the surface of the at least one eye component to draw out the cross-linking agent at or near the surface of the at least one eye component. The method also includes activating the cross-linking agent by delivering an initiating element to the at least one eye component.

According to yet another aspect of the present disclosure, a method of treating an eye includes applying an initial formulation including a cross-linking agent and a first active ingredient to a cornea to simultaneously open corneal epithelial tight junctions of the cornea and diffuse the cross-linking agent into the cornea. The method also includes, after applying the initial formulation, applying one or more secondary formulations including the cross-linking agent to the cornea to further diffuse the cross-linking agent into the cornea via the open corneal epithelial tight junctions. The application of the initial formulation and the one or more secondary formulations determines a concentration profile of the cross-linking agent in the cornea. Additionally, the method includes activating the cross-linking agent to initiate cross-linking in the cornea according to the concentration profile.

According to an aspect of the present disclosure, a system is disclosed for activating cross-linking in at least one eye component of an eye. The system includes a kit containing a first cross-linking agent, a second cross-linking agent, a third cross-linking agent and/or a reverse osmotic fluid, and a fourth formulation for closing epithelial tight junctions. Each aspect of the kit advantageously corresponds with a step in a method for activating cross-linking in at least one eye component of an eye.

According to additional aspects of the present disclosure, a kit for transepithelial delivery of a cross-linking agent to a cornea includes a first vessel containing an initial formulation that includes a cross-linking agent and a first active ingredient configured to open corneal epithelial tight junctions when applied to a cornea and one or more second vessels each containing a respective secondary formulation that includes the cross-linking agent for application to the cornea after the initial formulation is applied to the cornea.

These and other aspects of the present disclosure will become more apparent from the following detailed description of embodiments of the present disclosure when viewed in conjunction with the accompanying drawings.

### BRIEF DESCRIPTION OF THE DRAWINGS

FIG. 1 provides a block diagram of an example delivery system for delivering a cross-linking agent and an activator to a cornea of an eye in order to initiate molecular cross-linking of corneal collagen within the cornea.

FIG. 2A provides a flowchart showing an example embodiment according to aspects of the present disclosure for activating cross-linking within cornea tissue using a cross-linking agent and an initiating element.

FIG. 2B provides a flowchart similar to FIG. 2A where Riboflavin may be applied topically as the cross-linking agent and UV light may be applied as the initiating element.

FIG. 3 illustrates an example embodiment of an approach for stabilizing corneal structure by conveying a cross-linking agent to regions of a cornea using ultrasound to increase the permeability thereof.

FIG. 4 illustrates an example embodiment similar to FIG. 3 where microspheres are utilized in combination with the ultrasound to enhance the permeability of the corneal tissue.

FIG. 5 illustrates an example embodiment of an approach for stabilizing corneal structure by conveying a cross-linking agent to regions of a cornea using iontophoresis.

FIG. 6 illustrates an example embodiment similar to FIG. 5 where the cross-linking agent is dissolved in a solvent to enhance its permeability.

FIG. 7 illustrates an example embodiment of an approach for stabilizing corneal structure by conveying a cross-linking agent to regions of a cornea using a reverse osmotic fluid to draw the cross-linking agent away from the corneal surface.

FIG. 8 illustrates an example embodiment of an approach for stabilizing corneal structure by conveying a cross-linking agent to regions of a cornea using a neutral compound to increase pressure on the cross-linking agent.

FIG. 9A illustrates an example embodiment of an approach for stabilizing corneal structure by allowing a cross-linking agent to diffuse through the cornea during a period of time T.

FIG. 9B illustrates an example embodiment similar to FIG. 9A where the cross-linking agent is Riboflavin and the initiating element is UV light.

FIG. 10A illustrates an example embodiment of an approach for conveying a cross-linking agent to a region of a cornea by using a combination of one or more approaches discussed herein.

FIG. 10B illustrates yet another example approach for stabilizing changes in corneal structure according to aspects of the present invention.

FIG. 11 illustrates an example cross-linking treatment according to aspects of the present invention.

FIG. 12 illustrates the absorption spectrum for Riboflavin.

FIG. 13 illustrates photochemical reaction for Riboflavin providing pathway options.

FIG. 14A illustrates an example embodiment for controlling distribution of cross-linking agent in corneal tissue by generating heat in the corneal tissue according to aspects of the present invention.

FIG. 14B illustrates another example embodiment for controlling distribution of cross-linking agent in corneal tissue by directly controlling the temperature of the cross-linking agent according to aspects of the present invention.

FIG. 14C illustrates yet another example embodiment for controlling distribution of cross-linking agent in corneal tissue by additionally controlling the temperature of the corneal tissue according to aspects of the present invention.

FIG. 15 is a block diagram of an exemplary system for controlling the distribution of the cross-linking agent within the eye through permeability regulation system(s), temperature regulation system(s), and/or diffusion influencing compounds according to feedback information from feedback system(s).

FIG. 16 illustrates an example treatment for activating cross-linking in a cornea according to aspects of the present invention.

FIGS. 17A-C illustrate example kits that includes formulations for activating cross-linking in an eye according to aspects of the present invention.

### DETAILED DESCRIPTION

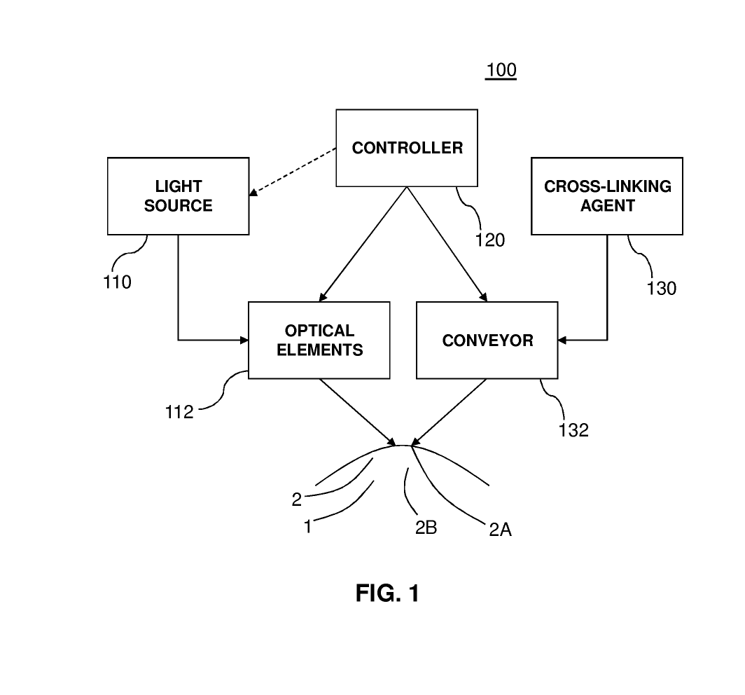

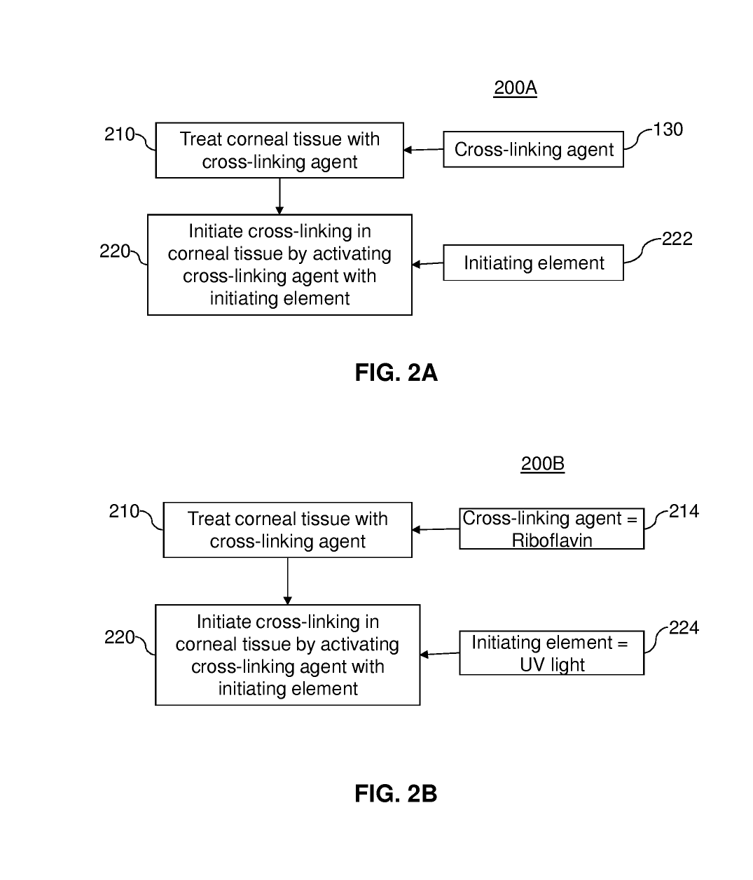

FIG. 1 provides a block diagram of an example delivery system 100 for delivering a cross-linking agent 130 and an initiating element (e.g. 222 shown in FIG. 2A) to a cornea 2 of an eye 1 in order to initiate molecular cross-linking of corneal collagen fibrils within the cornea 2. Cross-linking can stabilize corneal tissue and improve its biomechanical strength. The delivery system 100 includes a conveyor 132 for conveying the cross-linking agent 130 to the cornea 2. The cross-linking agent 130 can be conveyed to the cornea 2 by, for example, an eye dropper, a drug application device, such as a bandage, or can be included in a cream applied to the eye 1. The delivery system 100 includes a light source 110 and optical elements 112 for directing light to the cornea 2. The delivery system 100 also includes a controller 120 that is coupled to the conveyor 132 and the optical elements 112. The conveyor 132 may be an apparatus adapted to apply the cross-linking agent 130 according to particular patterns on the cornea 2 advantageous for causing cross-linking to take place within the corneal tissues. The conveyor 132 may apply the cross-linking agent 130 to a corneal surface 2A (e.g., an epithelium), or to other locations on the eye 1. Particularly, the conveyor 132 may apply the cross-linking agent 130 to an abrasion or cut of the corneal surface 2A to facilitate the transport or penetration of the cross-linking agent through the cornea 2 to a mid-depth region 2B. As described further herein, the conveyor 132 can be adapted to convey the cross-linking agent 130 to regions of the eye 1 including depths below the corneal surface 2A, by utilizing iontophoresis, reverse-osmotic fluids, neutral compounds, and/or by increasing the permeability of the cornea 2 using heat energy and/or ultrasound to modify the corneal structure and increase its permeability. Additionally or alternatively, the delivery system 100 can include one or more additional components (not shown) to convey the cross-linking agent 130.

For the sake of clarity, the non-limiting embodiments disclosed herein describe the operation of aspects of the present disclosure in conveying the cross-linking agent 130 to regions of the cornea 2. However, the system 100, and the embodiments disclosed herein for conveying the cross-lining agent 130 to regions of the eye 1, refer to systems and methods for directing the cross-linking agent 130 to regions of at least one eye component, and it is understood that the at least one eye component may include a cornea, a limbus, a sclera, and/or a retina.

As described below in connection with FIGS. 2A-2B, which describe an exemplary operation of the delivery system 100, the cross-linking agent 130 is applied to the cornea 2 using the conveyor 132. Once the cross-linking agent 130 has been applied to the cornea 2, the cross-linking agent 130 is initiated by the light source 110 (i.e. the initiating element) to cause cross-linking agent 130 to absorb enough energy to release Riboflavin and free oxygen radicals within the cornea 2. Once released, the Riboflavin and free oxygen radicals (i.e. singlet oxygen) form covalent bonds between corneal collagen fibrils and thereby cause the corneal collagen fibrils to cross-link and change the structure of the cornea 2. For example, activation of the cross-linking agent 130 with the light source 110 delivered to the cornea 2 through the optical elements 112 may result in cross-linking in the mid-depth region 2B of the cornea 2 and thereby strengthen and stiffen the structure of the cornea 2.

Although eye therapy treatments may initially achieve desired reshaping of the cornea 2, the desired effects of reshaping the cornea 2 may be mitigated or reversed at least partially if the collagen fibrils within the cornea 2 continue to change after the desired reshaping has been achieved. Indeed, complications may result from further changes to the cornea 2 after treatment. For example, a complication known as post-LASIK ectasia may occur due to the permanent thinning and weakening of the cornea 2 caused by LASIK surgery. In post-LASIK ectasia, the cornea 2 experiences progressive steepening (bulging).

Aspects of the present disclosure provide approaches for initiating molecular cross-linking of corneal collagen to stabilize corneal tissue and improve its biomechanical strength. For example, embodiments may provide devices and approaches for preserving the desired corneal structure and shape that result from an eye therapy treatment, such as LASIK surgery. In addition, aspects of the present disclosure may provide devices and approaches for initiating cross-linking at depths of the cornea 2 below the epithelium 2A by first conveying the cross-linking agent 130 to regions of the cornea 2 below the epithelium 2A. Advantageously, aspects of the present disclosure allow for conveying the cross-linking agent 130 to regions below the epithelium 2A of the cornea 2 without requiring removal of the epithelium 2A. As described herein, the devices and approaches disclosed herein may be used to preserve desired shape or structural changes following an eye therapy treatment by stabilizing the corneal tissue of the cornea 2. The devices and approaches disclosed herein may also be used to enhance the strength or biomechanical structural integrity of the corneal tissue apart from any eye therapy treatment.

Some approaches initiate molecular cross-linking in a treatment zone of the cornea 2 where structural changes have been induced by, for example, LASIK surgery. However, it has been discovered that initiating cross-linking directly in this treatment zone may result in undesired haze formation. Accordingly, aspects of the present disclosure also provide alternative techniques for initiating cross-linking to minimize haze formation. In particular, the structural changes in the cornea 2 are stabilized by initiating cross-linking in selected areas of corneal collagen outside of the treatment zone. This cross-linking strengthens corneal tissue neighboring the treatment zone to support and stabilize the actual structural changes within the treatment zone.

With reference to FIG. 1, the optical elements 112 may include one or more mirrors or lenses for directing and focusing the light emitted by the light source 110 to a particular pattern on the cornea 2 suitable for activating the cross-linking agent 130. The light source 110 may be an ultraviolet light source, and the light directed to the cornea 2 through the optical elements 112 may be an activator of the cross-linking agent 130. The light source 110 may also alternatively or additionally emit photons with greater or lesser energy levels than ultraviolet light photons. The light source 110 can be a laser light source, and may emit a slightly diverging beam of nearly collimated light. For eye safety reasons, it is desirable that implementations incorporating a laser light source as the light source 110 deliver a slightly diverging beam, and prevent the occurrence of a converging beam being applied to the eye 1. Implementations utilizing a laser light source as the light source 110 are desirably able to deliver light emitted by the light source 110 to the eye 1 at an intensity level that is largely insensitive to changes in a distance from the light source 110.

The delivery system 100 also includes a controller 120 for controlling the operation of the optical elements 112 or the conveyor 132, or both. By controlling aspects of the operation of the optical elements 112 and the conveyor 132, the controller 120 can control the regions of the cornea 2 that receive the cross-linking agent 130 and that are exposed to the light source 110. By controlling the regions of the cornea 2 that receive the cross-linking agent 130 and the light source 110, the controller 120 can control the particular regions of the cornea 2 that are strengthened and stabilized through cross-linking of the corneal collagen fibrils. Furthermore, by controlling the depth region that the cross-linking agent 130 is conveyed to, the controller 120 can control the depth of cross-linking activity within the corneal tissue. In an implementation, the cross-linking agent 130 can be applied generally to the eye 1, without regard to a particular region of the cornea 2 requiring strengthening, but the light source 110 can be directed to a particular region of the cornea 2 requiring strengthening, and thereby control the region(s) of the cornea 2 wherein cross-linking is initiated by controlling the regions of the cornea 2 that are exposed to the light source 110. In another implementation, the light source 110 can be directed generally to the eye 1, and the cross-linking agent 130 can be conveyed to particular region(s) of the cornea 2, and thereby control the region of the cornea 2 wherein cross-linking is initiated. In yet another implementation, both the cross-linking agent 130 and the initiating element 110 can be conveyed and/or directed to particular regions of the cornea 2, and thereby jointly control the region(s) of the cornea 2 wherein cross-linking is initiated.

The optical elements 112 can be used to focus the light emitted by the light source 110 to a particular focal plane within the cornea 2, such as a focal plane that includes the mid-depth region 2B. In addition, according to particular embodiments, the optical elements 112 may include one or more beam splitters for dividing a beam of light emitted by the light source 110, and may include one or more heat sinks for absorbing light emitted by the light source 110. The optical elements 112 may further include filters for partially blocking wavelengths of light emitted by the light source 110 and for advantageously selecting particular wavelengths of light to be directed to the cornea 2 for activating the cross-linking agent 130. The controller 120 can also be adapted to control the light source 110 by, for example, toggling a power switch of the light source 110.

In an implementation, the controller 120 may include hardware and/or software elements, and may be a computer. The controller 120 may include a processor, a memory storage, a microcontroller, digital logic elements, software running on a computer processor, or any combination thereof. In an alternative implementation of the delivery system 100, the controller 120 may be replaced by two or more separate controllers or processors. For example, one controller may be used to control the operation of the conveyor 132, and thereby control the precise rate of delivery, location of conveyance, depth of penetration, and/or concentration of the cross-linking agent 130 to the cornea 2. Another controller may be used to control the operation of the optical elements 112, and thereby control with precision the delivery of the light source 110 (i.e. the initiating element) to the cornea 2 by controlling any combination of: wavelength, bandwidth, intensity, power, location, depth of penetration, and duration of treatment. In addition, the function of the controller 120 can be partially or wholly replaced by a manual operation. For example, the conveyor 132 can be manually operated to deliver the cross-linking agent 130 to the cornea 2 without the assistance of the controller 120. In addition, the controller 120 can operate the conveyor 132 and the optical elements 112 according to inputs dynamically supplied by an operator of the delivery system 100 in real time, or can operate according to a pre-programmed sequence or routine.

Referring to FIG. 2A, an example embodiment 200A according to aspects of the present disclosure is illustrated. Specifically, in step 210, the corneal tissue is treated with the cross-linking agent 130. Step 210 may occur, for example, after a treatment is applied to generate structural changes in the cornea and produce a desired shape change. Alternatively, step 210 may occur, for example, after it has been determined that the corneal tissue requires stabilization or strengthening. The cross-linking agent 130 is then activated in step 220 with an initiating element 222. In an example configuration, the initiating element 222 may be the light delivered from the light source 110 shown in FIG. 1. Activation of the cross-linking agent 130, for example, may be triggered thermally by the application of microwaves or light.

As the example embodiment 200B of FIG. 2B shows further, Riboflavin may be applied topically as a cross-linking agent 214 to the corneal tissue in step 210. As also shown in FIG. 2B, ultraviolet (UV) light may be applied as an initiating element 224 in step 220 to initiate cross-linking in the corneal areas treated with Riboflavin. Specifically, the UV light initiates cross-linking activity by causing the applied Riboflavin to release reactive Riboflavin and oxygen radicals in the corneal tissue. In particular, the Riboflavin acts as a sensitizer to convert O₂ into singlet oxygen along with radical Riboflavin, causing cross-linking within the corneal tissue.

According to one approach, the Riboflavin may be applied topically to the corneal surface, and transepithelial delivery allows the Riboflavin to be applied to the corneal stroma. In general, the application of the cross-linking agent sufficiently introduces Riboflavin to mid-depth regions of the corneal tissue where stronger and more stable structure is desired.

In one example, a lower concentration of the cross-linking agent is broadly applied to the cornea, e.g., in the form of a drip, after a pattern of energy is applied. The lower concentration of the cross-linking agent is effective in activating cross-linking in the regions treated with energy, because these regions receive more cross-linking agent. Meanwhile, the effect on the other regions of the cornea may be insignificant due to the low concentration and the lower amount of cross-linking agent received by these other regions.

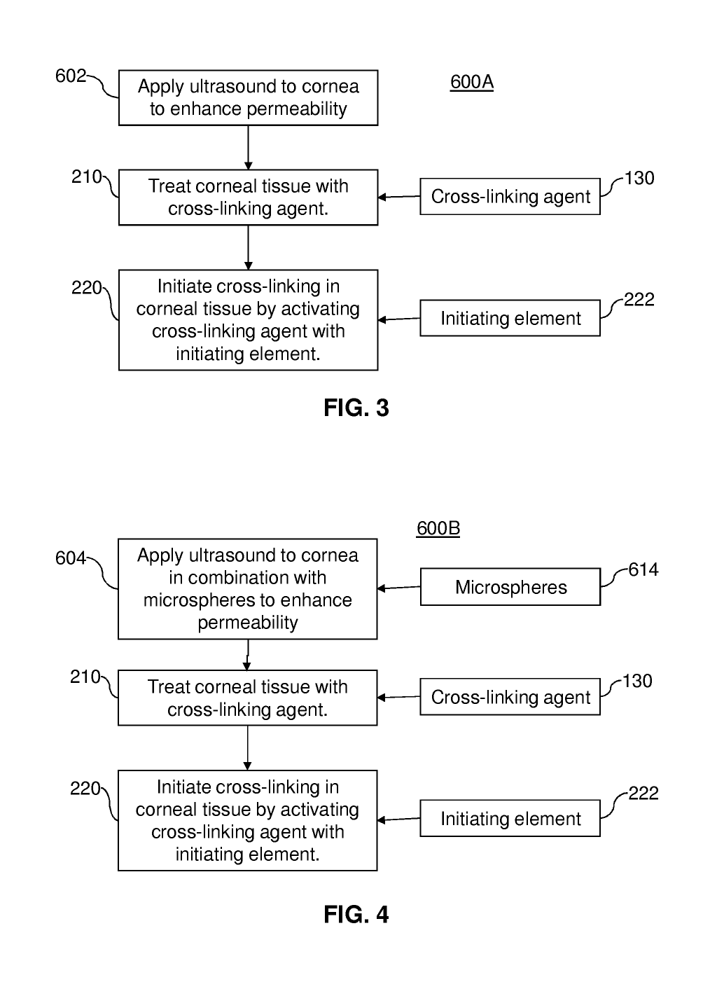

Referring to the embodiment 600A shown in FIG. 3, ultrasound is applied to the cornea 2 in step 602 before the cross-linking agent 130 is applied in step 210. Ultrasound produces minor structural changes in the epithelium 2A and enhances corneal permeability to the cross-linking agent 130. In one example, the ultrasound may be applied at a frequency of about 880 kHz with intensities in the range of about 0.19 to about 0.56 W/cm² in a continuous wave for about five minutes. However, it is understood that the ultrasound may be applied according to other parameters to achieve the desired level of corneal permeability. For example, permeability increases with increasing ultrasound intensity. In addition, the ultrasound may be delivered, alternatively or additionally, in a pulsed wave. Furthermore, the cross-linking agent 130 may be combined in varying concentrations with another agent, such as EDTA, benzalkonium chloride, or an alcohol, to promote further delivery across the epithelium 2A.

Referring to the embodiment 600B shown in FIG. 4, the effectiveness of the ultrasound may be enhanced by applying the ultrasound in combination with microspheres (or microbubbles) 614 as is shown in step 604 of the embodiment 600B. The microspheres 614 measure approximately 1 to 3 μm in diameter. In some embodiments, the microspheres 614 may be perflutren lipid microspheres, i.e., lipid coated spheres filled with octafluoropropane gas. The microspheres 614 are sufficiently small and stable to transition into the cornea 2. The microspheres exhibit lower acoustic impedance and allow the ultrasound energy applied to the cornea to be more effectively focused. As such, the ultrasound energy applied in step 604 may be reduced relative to the level applied in step 602 of embodiment 600A, while producing the desired increase in permeability of the cornea 2.

While the embodiments 600A and 600B each provide for increasing the permeability of the corneal tissues, in some embodiments, the cross linking agent 130 may be dissolved in a different carrier to promote delivery across the corneal surface 2A. For example, the cross-linking agent 130 may be combined in varying concentrations with another agent, such as EDTA, benzalkonium chloride, or an alcohol, to promote further delivery across the corneal surface 2A.

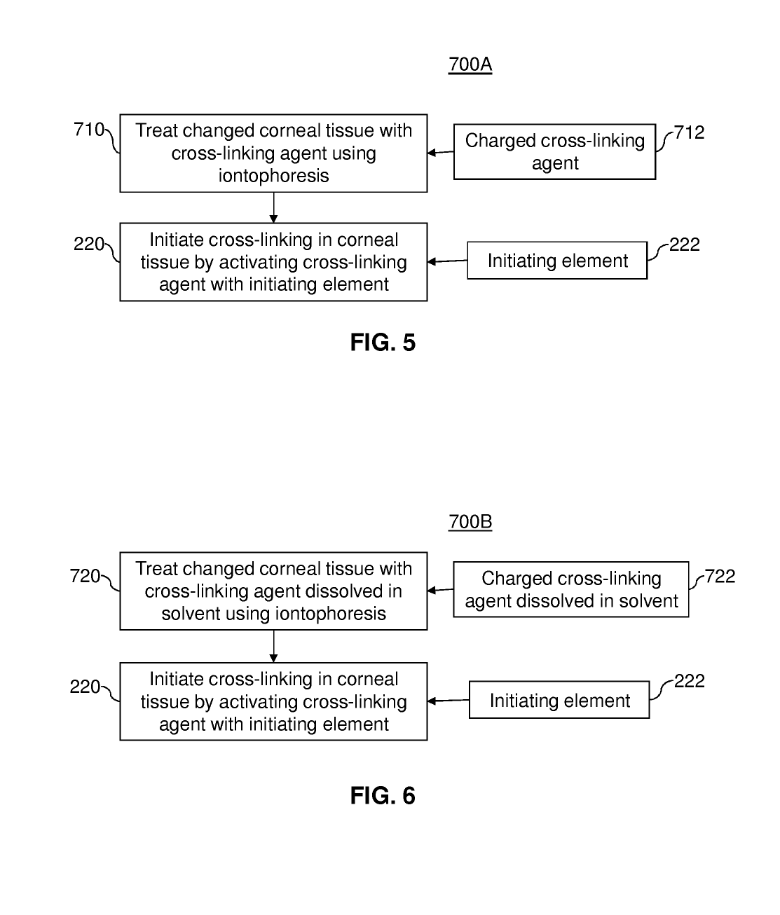

Referring to embodiment 700A shown in FIG. 5, iontophoresis is employed in step 710 to deliver a charged cross-linking agent 712 through the epithelium 2A to the corneal tissue below. In general, iontophoresis uses an electric charge to deliver the charged cross-linking agent 712 across the epithelium 2A. High concentrations of a charged cross-linking agent 712 may be delivered transdermally by repulsive electrical forces using a small electrical charge applied to an iontophoretic chamber containing the similarly charged cross-linking agent 712. The electrical force may be an electrostatic force or an electromotive force and may be applied according to continuous waves, pulsed waves, or any combination thereof.

Referring to embodiment 700B shown in FIG. 6, iontophoresis can be utilized to deliver a charged cross-linking agent dissolved in a solvent 722, which is shown in step 720. In the embodiment 700B, the iontophoretic chamber is filled with a solution containing the charged cross-linking agent 712 and its solvent. The solvent can be, for example, EDTA, benzalkonium chloride, or an alcohol, to promote further delivery across the corneal surface 2A.

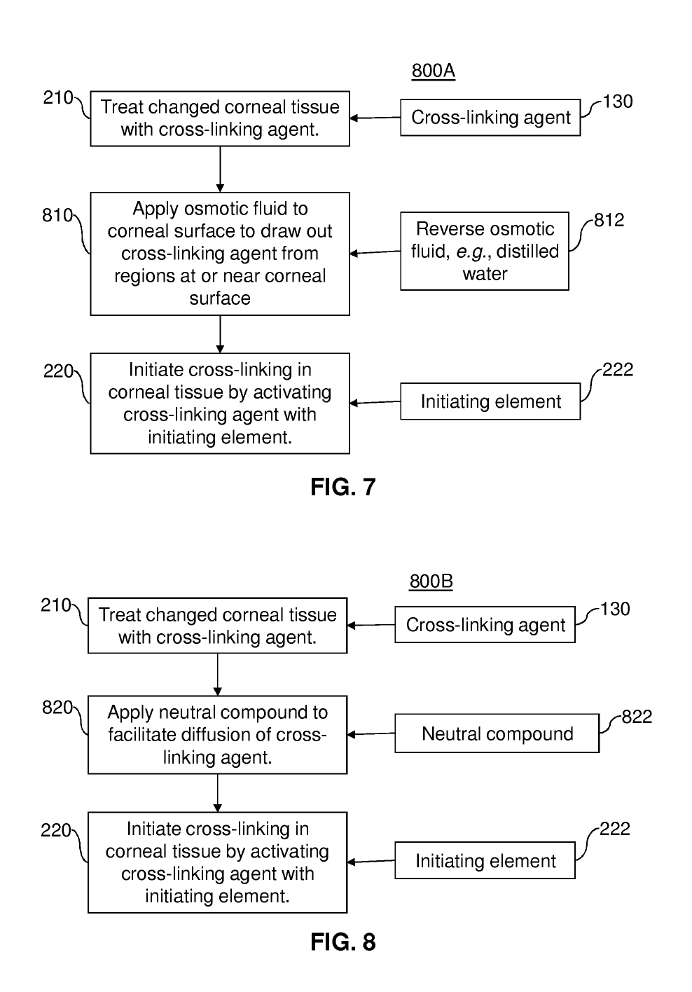

Referring to the embodiment 800A shown in FIG. 7, the amount of the cross-linking agent 130 at varying depths of the cornea 2 may also be controlled by applying a reverse osmotic fluid 812 to the surface of the cornea 2 to draw the cross-linking agent 130 from the corneal tissue, particularly from regions near the surface of the cornea 2A. The cross-linking agent 130 is applied topically to corneal tissue in step 210. A period of time T can then optionally be allowed to pass such that, during the period of time T, the cross-linking agent 130 diffuses from the corneal surface 2A into the underlying corneal structure according to an exponential gradient. Thus, the concentration of the cross-linking agent 130 in the corneal tissue is generally greater in regions closer to the surface (epithelium 2A). In some cases, it is desirable to have greater concentrations of the cross-linking agent 130 in regions farther below the corneal surface 2A relative to the concentrations of the cross-linking agent 130 in regions closer to the corneal surface 2A. For example, greater concentrations of the cross-linking agent 130 at greater depths may be more effective in stabilizing the corneal structure, whereas excessive cross-linking at or near the surface may create unwanted effects.

To achieve greater concentrations of Riboflavin farther below the surface, in step 810, the reverse osmotic fluid 812, such as distilled water, is applied to the corneal surface 2A. The reverse osmotic fluid 812 at the surface acts to draw the cross-linking agent 130 out from regions of corneal tissue closer to the corneal surface 2A. The concentration of the cross-linking agent 130 in the regions closer to the corneal surface 2A is then reduced relative to the regions farther below. Thus, the use of the reverse osmotic fluid 812 can produce a “reverse gradient” of the amount of the cross-linking agent 130 along a path moving deeper into the cornea 2.

The resulting distribution of the cross-linking agent 130 is then activated by the application of an initiating element 222, e.g., UV light, in step 220 to initiate cross-linking in the corneal regions treated with the cross-linking agent 130. The cross-linking activity generally corresponds to the distribution of cross-linking agent 130, which is produced in part by the application of the reverse osmotic fluid 812 in step 810.

Referring to the embodiment 800B shown in FIG. 8, a second (neutral) compound 822 may be applied after the cross-linking agent 130 is applied in step 820. The second compound 822 applies a pressure to the cross-linking agent 130 and promotes diffusion of the cross-linking agent 130 to depths of the cornea 2. The neutral compound 822 may be applied to the cornea 2 at any time during the embodiments described herein. For example, the second compound 822 may be applied at a time when diffusion of the cross-linking agent 130 has slowed and needs to be encouraged by the neutral compound.

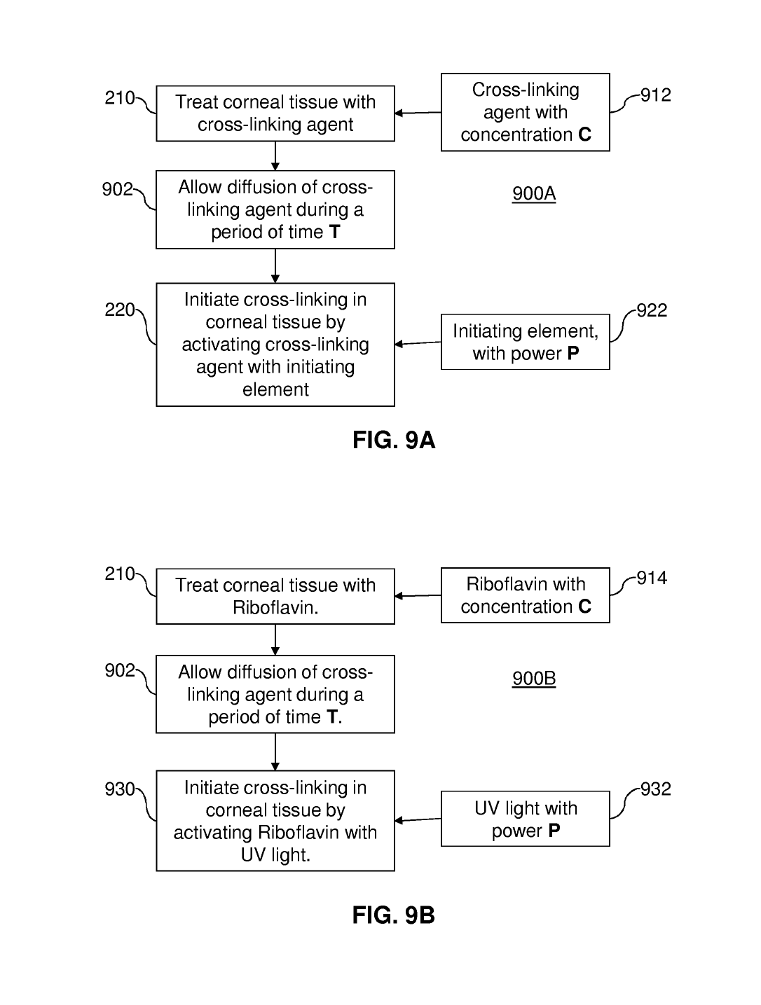

FIGS. 9A and 9B provide approaches for conveying a cross-linking agent to a region of the cornea 2 by controlling the concentration of the cross-linking agent and the time allowed for the cross-linking agent to diffuse before it is initiated.

Referring to FIG. 9A, an example embodiment 900A according to aspects of the present disclosure is illustrated. Specifically, in step 210, corneal tissue is treated with a cross-linking agent 912 with a concentration C. Step 210 may occur, for example, after a treatment is applied to generate structural changes in the cornea 2 and produce a desired shape change. The cross-linking agent 912 may be applied to the regions of the corneal tissue where the structural changes have occurred and/or to areas around the structural changes. The cross-linking agent 912, for example, may be applied topically to the epithelium 2A of the cornea 2. In step 902, a period of time T is allowed to pass. During the period of time T, the cross-linking agent 912 diffuses into the underlying corneal structure according to an exponential gradient. The distribution of cross-linking agent, i.e., concentration of cross-linking agent at depths at and below the epithelium, depends at least on the concentration C and the period of time T.

The cross-linking agent 912 is then activated in step 220 with an initiating element 922. Activation of the cross-linking agent 912 is triggered by the application of microwaves or light. As such, the initiating element 922 is applied with a power P. The power P of the initiating element 922 determines the extent to which the distribution of cross-linking agent 912 is activated. For example, an initiating element applied with a greater power P may reach greater depths below the epithelium 2A and allow the cross-linking agent to be activated at these depths. The parameters C, P, and T may be selected as independent variables to achieve the appropriate amount of cross-linking at desired depths of the cornea.

As the example embodiment 900B of FIG. 9B shows further, Riboflavin may be applied topically as a cross-linking agent 914 to corneal tissue in step 910. As also shown in FIG. 2, ultraviolet (UV) light is applied as an initiating element 932 in step 930 to initiate cross-linking in the corneal areas treated with Riboflavin. Specifically, the UV light 932 initiates cross-linking activity by causing the applied Riboflavin 914 to release reactive Riboflavin and oxygen radicals in the corneal tissue. In particular, the Riboflavin acts as a sensitizer to convert O₂ into singlet oxygen along with radical Riboflavin, causing cross-linking within the corneal tissue. According to one approach, the Riboflavin may be applied topically to the corneal surface 2A, and transepithelial delivery allows the Riboflavin to be applied to the corneal stroma.

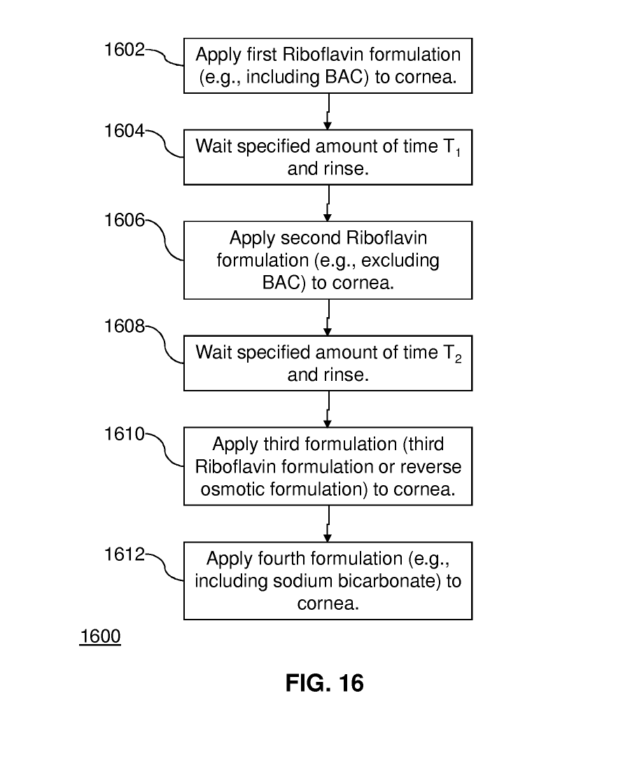

As illustrated in the example treatment 1600 of FIG. 16, the Riboflavin formulation may be developed for transepithelial delivery containing a specified amount of active and inactive ingredients. In addition to the Riboflavin, the ingredients, for example, may include benzalkonium chloride (BAC), which is known to open the tight junctions of the epithelium. As shown in FIG. 16, the Riboflavin formulation is applied to the cornea in step 1602. After waiting a specified amount of time T₁, e.g., approximately five minutes, the cornea is rinsed in step 1604. In step 1606, a second Riboflavin formulation is applied to the corneal epithelium. This second Riboflavin formulation does not contain BAC, but the Riboflavin freely diffuses through the now open epithelial tight junctions. After waiting a specified amount of time T₂, e.g., approximately five minutes, the cornea is rinsed in step 1608 and a third Riboflavin formulation or a reverse osmotic formulation is applied to the corneal epithelium in step 1610. After waiting a specified amount of time T₃, e.g., approximately five minutes, the cornea is rinsed. A fourth formulation, which may contain ingredients such as sodium bicarbonate, is then applied in step 1612 to close the tight junctions. Advantageously, the method 1600 may reduce post-operative pain and alleviate the occurrence of small erosions in the epithelium associated with prolonged exposure to BAC.

According to some aspects of the present disclosure, the amount of time that is waited for one or more of the steps of the treatment method 1600 (e.g., the steps 1604, 1608) can be determined based on a monitoring of the eye for the presence and distribution of the cross-linking agent. Exemplary systems and methods for monitoring the eye for the presence and distribution of the cross-linking agent are disclosed in U.S. application Ser. No. 13/488,097, filed Jun. 4, 2012, the contents of which is herein incorporated by reference in its entirety. According to additional and/or alternative aspects of the present disclosure, the amount of time that is waited for one or more of the steps of the treatment method 1600 can be determined based on a monitoring of the activation of the cross-linking agent in the cornea. Exemplary systems and methods for monitoring the activation of the cross-linking agent in the cornea are disclosed in U.S. application Ser. No. 13/051,699, filed Mar. 18, 2011, the contents of which is herein incorporated by reference in its entirety.

It should be understood that the exemplary treatment method 1600 can omit steps, include additional steps, and/or modify the order of steps presented above. For example, the treatment method 1600 can include applying an initial formulation including a cross-linking agent and a first active ingredient to a cornea to simultaneously open corneal epithelial tight junctions and diffuse the cross-linking agent into the cornea (e.g., at step 1602), applying one or more secondary formulations including the cross-linking agent (and omitting the first active ingredient) to the cornea after the initial formulation to further diffuse the cross-linking agent into the cornea via the open corneal epithelial tight junctions (e.g., at step 1606 and/or 1610), optionally applying a reverse osmotic substance to the cornea to draw the cross-linking agent from the cornea (e.g., at step 1610), and optionally applying a final formulation including the cross-linking agent and a second active ingredient after the one or more secondary formulations to close the corneal epithelial tight junctions (e.g., at step 1612), and activating the cross-linking agent to initiate cross-linking in the cornea (e.g., as described for a steps 220 and 930 with respect to FIGS. 2A-10B). The treatment method 1600 can further include waiting specified amounts of time and/or rinsing the cornea between the application of any and/or each of the formulations (e.g., at steps 1604, at step 1606, and/or between steps 1610 and 1612). As described below, the application of such formulations (and optionally waiting for specified amounts of time between such applications) determines a concentration profile of the cross-linking agent, which when activated determines a profile of cross-linking of the corneal tissue. As another example, the step of activating the cross-linking agent can include one or more applications of an initiating element after all of the formulations have been applied and/or after some of the formulations have been applied.

It also should be understood that, while the above exemplary treatment method 1600 is described as including formulations including Riboflavin, in additional and/or alternative aspects, the treatment methods 1600 can include one or more formulations containing other types of cross-linking agents (e.g., Rose Bengal and/or quinoxalines). Also, while the above exemplary treatment method 1600 is described as including BAC and/or sodium bicarbonate as active ingredients in the initial formulation and the final formulation, respectively, it should be understood that one or more of the initial, secondary, and/or final formulations can include other types of active and/or inactive ingredients. For example, additional active ingredients (such as, e.g., 1,2-Dihydro-6,7-dimethyl1-2-keto-1-D-ribityl-3-quinoxalinecarboxylic acid monohydrate) and/or inactive ingredients that can be included in the initial formulation(s), the secondary formulation(s), and/or the final formulation(s) to enhance or otherwise controllably affect the cross-linking in the cornea are disclosed in U.S. Provisional Application No. 61/825,072, filed on May 19, 2013, and U.S. Provisional Application No. 61/895,008, filed on Oct. 24, 2013, the contents of these applications being incorporated herein by reference in their entirety. It is contemplated that the composition of the formulations utilized in the treatment method 1600 can be predetermined and/or dynamically determined during treatment based on systems and methods for monitoring the eye for the presence and distribution of the cross-linking agent as disclosed in U.S. application Ser. No. 13/488,097, filed Jun. 4, 2012, the contents of which is herein incorporated by reference in its entirety, and/or systems and methods for monitoring the activation of the cross-linking agent in the cornea as disclosed in U.S. application Ser. No. 13/051,699, filed Mar. 18, 2011, the contents of which is herein incorporated by reference in its entirety. Additionally, it should be understood that the treatment method 1600 can include a different number of applications of the initial, secondary, and final formulations than that described above. In general, the treatments described herein may apply various formulations to control aspects of cross-linking activity, e.g., chemical activity, that occur at different times during the treatments.

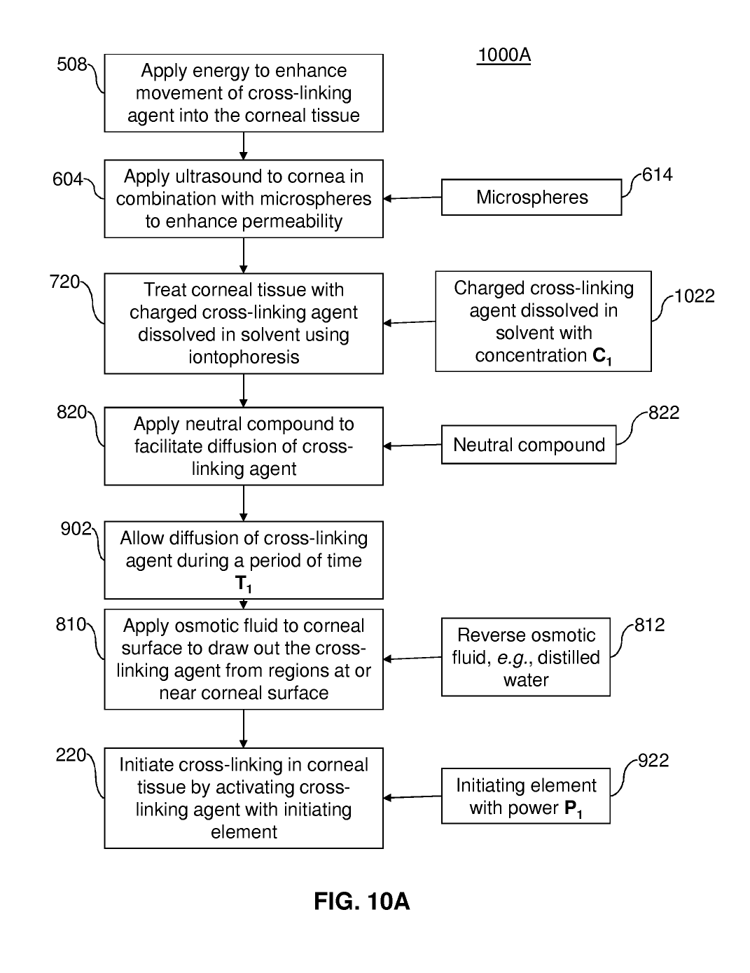

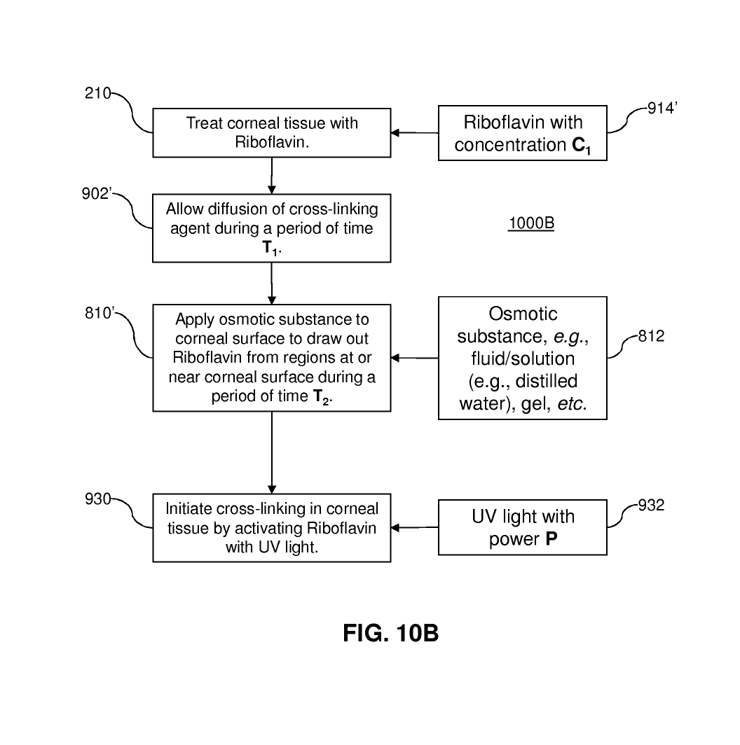

FIG. 10A provides a flowchart according to an example embodiment 1000A illustrating an approach for conveying the cross-linking agent to regions of the cornea 2 according to a combination of several different approaches previously discussed. Thus, the embodiment 1000A illustrates that the various systems and approaches discussed herein can be used in any combination or subset to precisely convey the cross-linking agent 130 to regions of the cornea 2 and thereby control the regions of the cornea 2 where cross-linking is initiated and the biomechanical strength is increased. In step 508, energy is applied to the cornea to enhance the movement of the cross-linking agent 130 into the corneal tissue (e.g., increase the permeability of the cornea 2 to the cross-linking agent). In step 604, ultrasound is applied to the cornea 2 in combination with microspheres 614 that focus the acoustic energy and enhance the increase in permeability due to the ultrasound. In step 720, the corneal tissue is treated with a charged cross-linking agent 1022 dissolved in a solvent with a concentration C₁. Also in step 720, the charged cross-linking agent is urged to penetrate beyond the epithelium using iontophoresis. In step 820, a secondary (neutral) compound 822 is applied to the eye to facilitate diffusion of the cross-linking agent through the epithelium 2A. In step 902, the cross-linking agent is allowed to diffuse into the corneal tissue during a period of time T₁. In step 810, a reverse osmotic fluid 812 is applied to the corneal surface 2A to draw the cross-linking agent away from regions at or near the corneal surface 2A. In step 220, the cross-linking agent thus distributed is activated with an initiating element 922 having a power P₁.

While the embodiment 1000A shown in FIG. 10A is shown as a flowchart, it is understood that the order of the steps as provided is not critical and many variations are possible. Furthermore, the embodiment 1000A may be further modified such that the entire procedure or portions thereof are carried out repeatedly and/or iteratively so as to gradually convey the cross-linking agent to regions of the cornea 2. In iterative implementations of the embodiment 1000A, each iteration of the steps (508, 604, 720, 820, 902, 810) can be identical, can be adjusted according to predetermined or dynamically determined information, or can be omitted. For example purposes: the microspheres 614 may be applied in combination with the ultrasound treatment on the first iteration of the embodiment 1000, but omitted on subsequent iterations; the concentration C₁ of the cross-linking agent 1022 can be adjusted to a concentration C₂, C₃, . . . Cₙ on subsequent iterations; similarly, the period of time T₁ in step 902 can be adjusted to times T₂, T₃, . . . Tₙ; the power P₁ of the initiating element 922 can be adjusted to powers P₂, P₃, . . . Pₙ; the step 810 can only be applied on a final iteration; the pattern and intensity with which the energy is applied in step 508 and/or the ultrasound is applied in step 604 can each be adjusted on each iteration, the order of the steps can be adjusted from one iteration to the next, etc. In addition, steps similar to the step 902 which allow for waiting for a period of time can be added between any of the steps in the embodiment 1000A and the period of time between each step for each iteration can vary according to predetermined or dynamically determined information.

Referring to FIG. 10B, another embodiment 1000B is presented to illustrate another combination of at least some of the embodiments shown in FIGS. 5-9. A cross-linking agent, i.e., Riboflavin, is applied topically as a cross-linking agent 914′ to corneal tissue in step 210 with a solution having concentration C₁. For example, the concentration C₁ can be a solution with 0.25% Riboflavin.

In step 902′, a period of time T₁ is allowed to pass. As described previously, during the period of time T₁, the Riboflavin diffuses from the corneal surface into the underlying corneal structure according to an exponential gradient. Thus, the concentration of Riboflavin in the corneal tissue is generally greater in regions closer to the surface. In some cases, it is desirable to have greater concentrations of Riboflavin in regions farther below the surface relative to the concentrations of Riboflavin in regions closer to the surface. For example, greater concentrations of Riboflavin at greater depths may be more effective in stabilizing the corneal structure, whereas excessive cross-linking at or near the surface may create unwanted effects.

To achieve greater relative concentrations of Riboflavin farther below the surface, in step 810′, a reverse osmotic substance 812, such as distilled water, is applied to the corneal surface for a period of time T₂. The reverse osmotic substance 1718 at the surface acts to draw the Riboflavin out from regions of corneal tissue closer to the surface. The concentration of the Riboflavin in the regions closer to the surface is then reduced relative to the regions farther below. Thus, the use of the reverse osmotic substance 318 can produce a “reverse gradient” of the Riboflavin concentration traveling in to the corneal tissue.

It is understood that distilled water is merely one example of a reverse osmotic substance 812 and other hypotonic fluids/solutions with appropriate concentrations may be employed. Alternatively, the reverse osmotic substance 812 may be a gel, hydrogel, a semisolid substance, or other non-liquid substance that can draw the cross-linking agent 914′ out of the corneal tissue.

The resulting distribution of Riboflavin is then activated by the application of an initiating element 932, i.e., UV light, in step 930 to initiate cross-linking in the corneal regions treated with Riboflavin. The cross-linking activity generally corresponds to the distribution of Riboflavin produced in part by the application of the reverse osmotic substance 812.

While described as first concentration C1 and durations T1 and T2, the embodiment 1000B can be carried out in an iterative scheme with each iteration having distinct values of concentration, duration(s), power, etc. (similar to the discussion in connection with the embodiment 1000A of FIG. 10A). In general, the cross-linking agent 914′ and reverse osmotic substances 812 may be applied in any number and combination of steps according to varying concentrations C and periods of time T to achieve a desired concentration profile in the corneal tissue. The desired concentration profile then determines the profile of cross-linking activity. For example, in the embodiment of FIG. 10B, Riboflavin may be applied topically in a solution with a different concentration C₃ between steps 810′ and 930. The diffusion of cross-linking agent 914′ in the corneal tissue follows Fick's laws of diffusion. As such, different concentration gradients may be generated in the corneal tissue causing the cross-linking agent to be drawn into or out of given depths of corneal tissue.

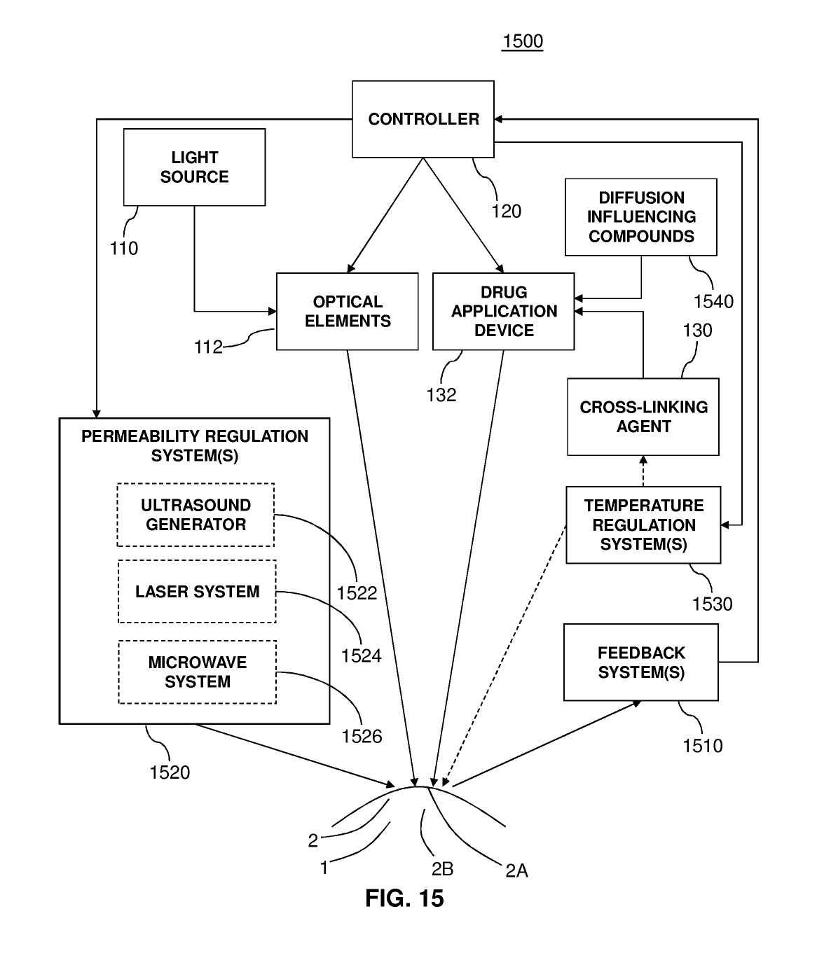

Further, in an iterative implementation of the embodiment 1000A or 1000B, one or more of the steps (508, 604, 720, 820, 902, 810, etc.) can be adjusted according to feedback information indicative of the progress of cross-linking in strengthening the corneal tissue. In an example embodiment, feedback information can be supplied by a feedback system (e.g., the feedback system(s) 1510 described in FIG. 15) configured to dynamically monitor cross-linking in the corneal tissue and provide an output signal indicative of a biomechanical strength of the corneal tissue, of an absorption of cross-linking agent within the corneal tissue, of corneal topography and/or pachymetry, etc. The feedback system can include an interferometer dynamically monitoring a three-dimensional surface profile of the surface of an eye and determining a biomechanical strength of the corneal tissue based on an amount of dynamic deformation of the surface profile of the eye due to, for example, changes in intraocular pressure corresponding to a cardiac cycle. The feedback system can alternatively or additionally include an OCT system or Scheimpflug system configured to dynamically characterize the response of corneal tissue to subtle perturbations and thereby determine the biomechanical strength of the corneal tissue.

The absorption of Riboflavin follows Beer's law at higher concentrations (typically near the surface of the cornea) and less so at greater depths in the corneal tissue where the concentration of Riboflavin becomes smaller. Such results indicate that unsafe concentrations of Riboflavin are less likely to reach the endothelium than was previously understood from misleading spectrophotometric data. In particular, the noise limit of the spectrophotometer led to the incorrect conclusion that there was a saturation point for the Riboflavin. However, with a clearer understanding of how Riboflavin is received by the corneal tissue, there is a need for systems and methods that can modify/tailor the concentration gradient according to aspects of the present disclosure described above. For example, some embodiments of the present disclosure provide for a system configured to distribute Riboflavin agent within corneal tissue without accounting for a saturation point of the Riboflavin.

In sum, embodiments stabilize a three-dimensional structure of corneal tissue through controlled application and activation of cross-linking in the corneal tissue. For example, the cross-linking agent and/or the initiating element are applied in a series of timed and controlled steps to activate cross-linking incrementally. Moreover, the delivery and activation of the cross-linking agent at depths in the cornea 2 depend on the concentration(s) of the cross-linking agent and the power(s) of the initiating element.

Although cross-linking agents, such as Riboflavin, may be effectively applied to the stroma by removing the overlying epithelium before application, it has been shown that cross-linking agents can chemically transition across the epithelium into the stroma. Indeed, Riboflavin may also be delivered to the stroma by applying it topically on the epithelium. Moreover, in some cases, the epithelium may be treated to promote the transition of the cross-linking agent through the epithelium. Accordingly, in the embodiments described herein, no removal of the epithelium is required. Advantageously, this eliminates the post-operative pain, healing period, and other complications associated with the removal of the epithelium.

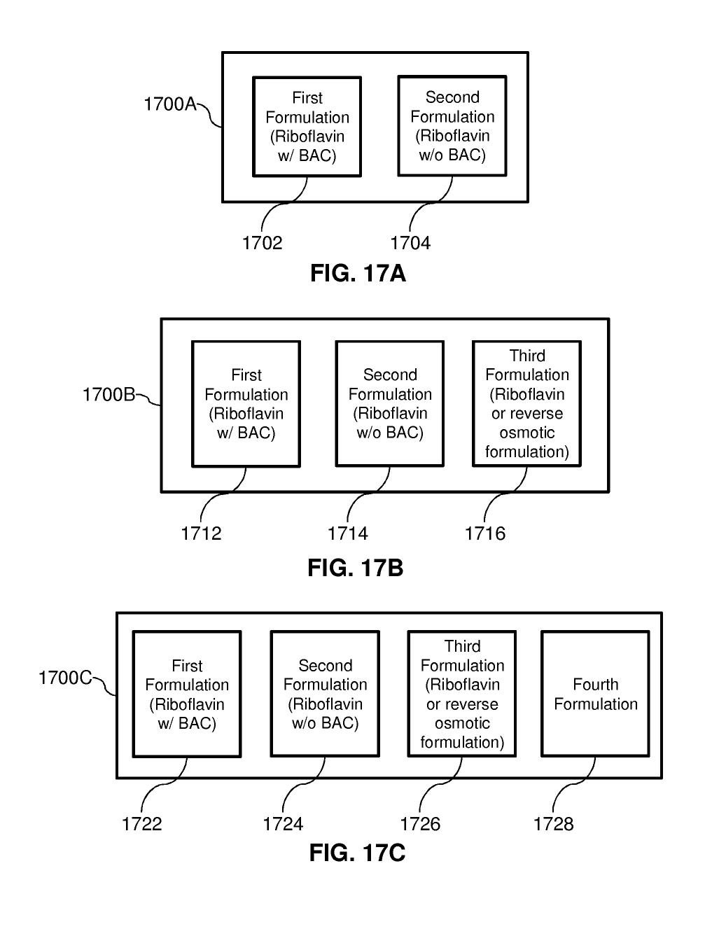

As shown in FIGS. 17A-C, the cross-linking agent and other formulations described herein may be packaged together in specific kits. For example, a kit may include the formulations that correspond to the steps of a treatment, e.g., the treatment 1600 illustrated and described with respect to FIG. 16. In one example, the transepithelial kit 1700A of FIG. 17A includes a first formulation 1702 containing Riboflavin (e.g., approximately 0.25%) and BAC (approximately 0.02%) for opening the tight junctions and a second formulation 1704 containing just Riboflavin (e.g., approximately 0.25%). In another example, the transepithelial kit 1700B includes a first formulation 1712 containing Riboflavin and BAC for opening the tight junctions, a second formulation 1714 containing just Riboflavin, and a third formulation 1716 containing a third Riboflavin formulation or a reverse osmotic formulation. In yet another example, the transepithelial kit 1700C may include a first formulation 1722 containing Riboflavin and BAC for opening the tight junctions, a second formulation 1724 containing just Riboflavin, a third formulation 1726 containing a third Riboflavin formulation or a reverse osmotic formulation, and a fourth formulation 1728 that closes the tight junctions. As illustrated in FIGS. 17A-17C, each of the formulations can be provided in one more vessels including, for example, the vessels for delivering a cross-linking agent described above (e.g., a dropper, syringe, other drug application device, an ointment applicator, a vial, or the like).

Specific kits are not limited to the particular first, second, third, or fourth formulations described herein, but may contain any number of different combinations. Thus, in general, the kits can include an initial formulation that includes the cross-linking agent and a first active ingredient configured to open corneal epithelial tight junctions when applied to a cornea, one or more secondary formulations that include the cross-linking agent (and omit the first active ingredient) for application to the cornea after the initial formulation is applied to the cornea, an optional formulation that includes a reverse osmotic substance, and, optionally, a final formulation that includes the cross-linking agent and a second active ingredient configured to close the corneal epithelial tight junctions when applied to the cornea after the initial formulation and the one or more secondary formulations. The kits can also optionally include one or more vessels containing a rinsing substance (e.g., saline) for rinsing the cornea between applications of the initial formulation(s), the secondary formulation(s), the reverse osmotic substance(s), and/or the final formulation(s). It should be understood that the kits can include formulations additionally and/or alternatively containing other types of cross-linking agents (e.g., Rose Bengal or quinoxalines) and/or other types of active and/or inactive ingredients (as described above) according to aspects of the present disclosure. Additionally, it should be understood that the kits can include a different number of the initial, secondary, and final formulations described in the examples above. The kits may include instructions that correspond to the steps of the methods described herein.

Although embodiments of the present disclosure may describe stabilizing corneal structure after treatments, such as LASIK surgery, it is understood that aspects of the present disclosure are applicable in any context where it is advantageous to form a stable three-dimensional structure of corneal tissue through cross-linking. For example, due to the advantageous structural changes caused by the cross-linking agent, the cross-linking agent may be applied as the primary aspect of some treatments. For example, a cross-linking agent may be applied to treat keratoconus.

In addition to regulating distribution and/or concentration of cross-linking agent during initiation of cross-linking so as to control the pattern of resulting cross-linking in the tissue, aspects of the present disclosure provide systems and methods for controlling the concentration of cross-linking agent within tissue following treatment. For example, aspects of the present disclosure provide better control of corneal cross-linking and prevent unintended cross-linking and/or other local phototoxicity by safely inhibiting, deactivating, or destroying, i.e., quenching, any residual Riboflavin that may remain in the corneal tissue after the desired cross-linking activity has been achieved. By quenching residual Riboflavin, aspects of the present disclosure improve patient safety and provide more accurate control of corneal cross-linking activity. It should be understood that, according to some aspects, the quenching substance can be provided in one or more vessels and included in the transepithelial kits (e.g., the transepithelial kits 1700A, 1700B, 1700C) described above. It should also be understood that, according to some aspects, the treatment method 1600 described above can include applying a quenching substance to the cornea to minimize photoactivation and unwanted cross-linking.

It has been observed that corneal cross-linking is an inefficient process, in which amounts of the cross-linking agent, e.g., Riboflavin, applied to the corneal tissue may not react with UV light during the cross-linking treatment. In addition, cross-linking treatments may attempt to achieve a desired depth and degree of cross-linking activity, for example, by limiting the amount of UV light directed at the cornea after Riboflavin has been applied or by limiting the potential for localized or regional patterning of cross-linking Limiting the activation of cross-linking activity during a treatment may result in applied Riboflavin remaining unreacted.

Accordingly, residual Riboflavin may remain in the corneal tissue after the treatment. Because Riboflavin's absorption spectrum includes a portion of the visible spectrum, environmental light can excite Riboflavin so as to serve as an initiating element even in the absence of an application of UV light. Residual Riboflavin may result in additional, unwanted cross-linking activity when the cornea is exposed to environmental light. Such problems have been addressed in photodynamic therapy by minimizing exposure to environmental light, e.g., keeping the patient in a dark room, until residual photodynamic agents either degrade or migrate from the treatment area. Riboflavin in the absence of light is generally regarded as safe (“GRAS”) and over time migrates out of the cornea and eye by diffusion, active transport, circulation, etc. However, aspects of the present disclosure provide systems and methods for quenching Riboflavin remaining in the eye following treatment and thereby prevent undesired cross-linking activity from occurring.

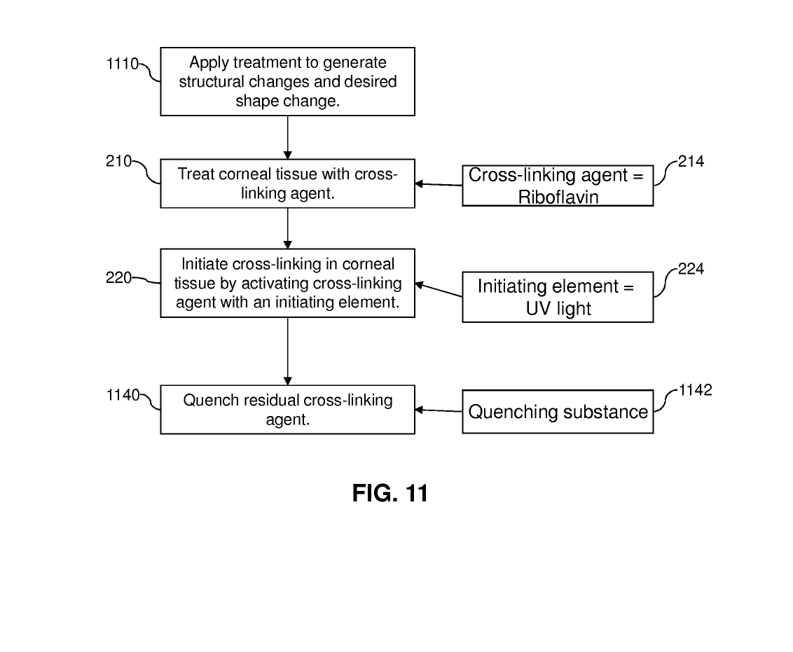

Referring to FIG. 11, a treatment, such as LASIK surgery, is applied in step 1110 to generate structural changes in the cornea and produce a desired shape change. In step 210, the corneal tissue is treated with a cross-linking agent 214, i.e., Riboflavin. Following step 1110, and before commencing instillation of the cross-linking agent in step 210, a time period with duration T is allowed to pass. The duration of the period T can be on the scale of hours, days, or weeks. In some examples, the corneal tissue 2 is allowed to settle/relax in its modified structural configuration during the time period T prior to initiating cross-linking to strengthen/stiffen the corneal tissue 2. In some examples, the duration of the period T can be selected based on measured and/or predicted biomechanical characteristics of the cornea (e.g., indicators of biomechanical strength before or after treatment 1110, corneal thickness, corneal topography, etc.) and/or based on patient data (e.g., age, health conditions, etc.).

In step 210, the cross-linking agent may be applied directly on the treated tissue and/or in areas around the treated tissue. In some embodiments, the cross-linking agent may be an ophthalmic solution that is broadly delivered by a dropper, syringe, other drug application device, or the like. Alternatively, the cross-linking agent may be selectively applied as an ophthalmic ointment with an appropriate ointment applicator. The cross-linking agent 214 is then activated in step 220 with an initiating element 224. In particular, photoactivation of the cross-linking agent 214 is triggered by the application of UV light from a corresponding energy or light source. The UV light initiates cross-linking activity by exciting the applied Riboflavin to generate reactive Riboflavin radicals and reactive oxygen radicals in the corneal tissue. Thus, the Riboflavin acts as a sensitizer to radical Riboflavin and to convert O₂ into singlet oxygen which causes cross-linking within the corneal tissue. The resulting cross-linking of and between collagen fibrils provides resistance to changes in corneal structure, thereby increasing strength.

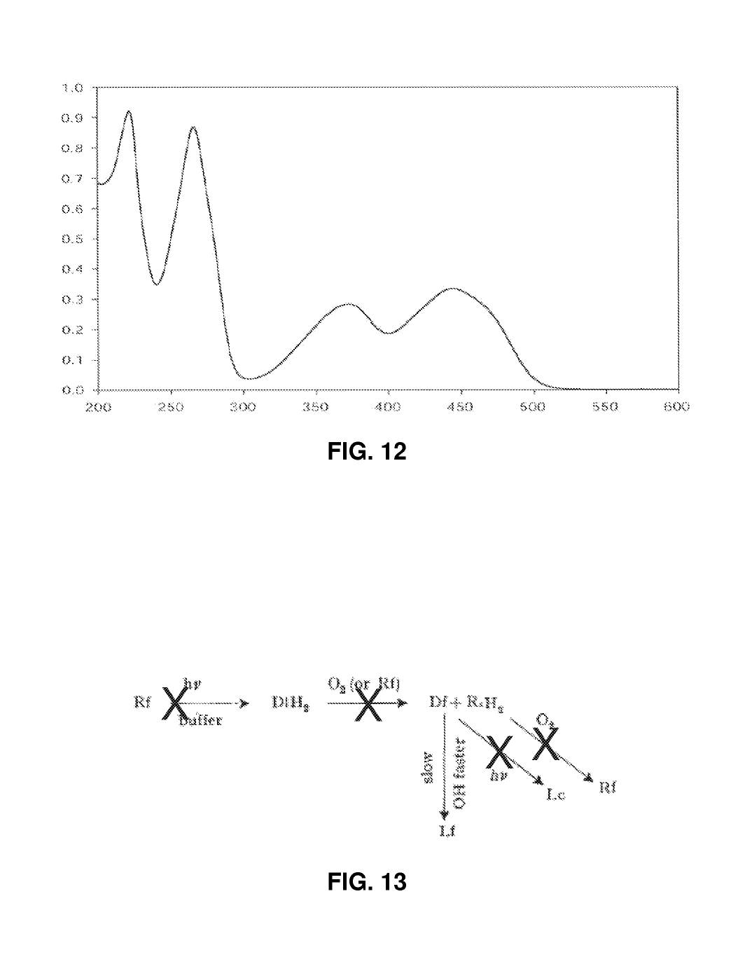

Referring to FIG. 12, the photodynamic action spectrum of Riboflavin appears to mimic its absorption spectrum, extending from approximately 266 nm to approximately 575 nm and including a portion of the visible spectrum, e.g., the peak at approximately 450 nm. While the quantum efficiency drops with increasing wavelength, Riboflavin can generally become excited by light absorbed throughout the visible spectrum. Because the absorption spectrum of Riboflavin includes a portion of environmental light (e.g., due to solar emissions transmitted through the Earth's atmosphere, which includes the visible spectrum from about 300 nm to about 900 nm), Riboflavin can be photoactivated by exposure to daylight and other visible sources of appropriate wavelength and intensity.

Examples of photoactivation of Riboflavin to thereby cause the formation of cross-links in fibril tissue are described further below, including Type I and Type II photoactivation mechanisms. Generally, Riboflavin absorbs energy from incident light according to the absorption spectrum shown in FIG. 12 and enters an excited state. The excited Riboflavin molecule decays by fluorescence to release the absorbed energy and decay into additional molecules, which process can release singlet oxygen and/or radical compounds. The singlet oxygen and/or radical compounds lead to cross-linking activity within the fibril tissue. Thus, aspects of the present disclosure refer to quenching agents as compounds which diminish the fluorescence of Riboflavin. Diminishing the fluorescence of Riboflavin corresponds to diminishing the generation of singlet oxygen and/or radical compounds and therefore corresponds to diminishing cross-linking activity. Compounds and substances that quench Riboflavin and those which prevent Riboflavin from being photoactivated to generate cross-linking activity.

Aspects of the present disclosure provide more effective control of cross-linking activity to minimize, for example, photoactivation of residual Riboflavin by exposure to environmental light. In particular, embodiments allow Riboflavin to be photoactivated when desired, titrated to a desired endpoint, and quenched with a selected substance to prevent unwanted cross-linking activity from residual Riboflavin. Referring again to FIG. 11, step 1140 applies a quenching substance 1142 (described further below) to the residual Riboflavin to minimize further photoactivation and unwanted cross-linking activity. The quenching substance 1142 (“quenching agent”) can be phenolic compounds, ascorbic acid, metallic ions, etc., to inhibit the photochemical reaction by, for example diminishing the fluorescence of Riboflavin.

FIG. 13 illustrates photochemical reaction for Riboflavin providing pathway options for inhibiting the photochemical reaction, described further below. The substance used to quench Riboflavin may be ascorbic acid, phenolic compounds, or a variety of metal ions. Other quenching substances may be common food and pharmaceutical compounds. According to aspects of the present invention, the quenching substance used to is pharmacologically safe at the intended (required) doses. In addition, the interaction to quench the Riboflavin may induce additional cross-linking activity by the quenching agent partially activating the Riboflavin. Furthermore, the interactions with Riboflavin do not produce byproducts in toxic concentrations.

Practically, Riboflavin-5-phosphate (also known as Flavin Mononucleotide (FMN)) is used for pharmaceutical preparations of Riboflavin. The photochemical and biological behavior of FMN is substantially identical to those of Riboflavin, but FMN is more soluble in water. The fluorescence of a solution of FMN may be quenched by the addition of purines or adenosine. Complexing FMN with a protein, as in most flavoproteins, abolishes the fluorescence. This type of fluorescence quenching is not necessarily associated with changes in absorption spectra, and arises because the complex formed is nonfluorescent. Heating the solution dissociates the complex, and the fluorescence therefore increases with temperature.

Ascorbic acid quenches both excited triplet Riboflavin and singlet oxygen (type I and type II mechanisms). The addition of ascorbic acid can protect Riboflavin oxidation in foods exposed to light. Ascorbic acid appears to inhibit photo-degradation of Riboflavin in a dose-dependent manner. When combined with greater than 40 mM ascorbic acid, more than 90% of Riboflavin remains unreacted after 4 hours of light exposure. This suggests that ascorbic acid does not chemically degrade Riboflavin, but blocks the light reaction. Ascorbic acid is also a potent anti-oxidant, effectively scavenging singlet oxygen which may be produced. Ascorbic acid preparations have been used topically to improve wound healing after laser refractive surgery in concentrations of approximately 10% ascorbic acid in distilled water.

Phenol and terpine are phenolic compounds found in food products, including beer. Phenolic compounds present in beer were shown by fluorescence spectroscopy and laser flash photolysis to deactivate both singlet- and triplet-excited states of Riboflavin with bimolecular rate constants close to the diffusion control. Fairly high concentrations of these compounds (greater than 0.3 M) are required for quenching, however, at concentrations of 0.36 M, greater than 90% of triplet-state is inhibited. A variety of phenolic compounds are extracted from plants and used as pharmaceuticals. This class of compounds provides a number of options for Riboflavin quenching. Other aromatic organic compounds which quench Riboflavin fluorescence include diphenols, purines, and pyrimidines.

Flavoproteins are a class of proteins containing Riboflavin or its cousins FMN or flavin adenine dinucleotide (FAD). Their function is to bind and transport Riboflavin in an inert state until it reaches its functional target. There is a large class of these compounds which perform the transport role in eukaryotic cells.

Accordingly, aspects of the present disclosure may apply appropriate preparations of any of the quenching substances described herein to provide better control of corneal cross-linking and prevent unintended cross-linking and/or other local phototoxicity produced from any residual Riboflavin. Examples are described herein, but it is understood that aspects of the present disclosure can generally be applied to inhibit R*, O₂*, and/or any excited intermediates, as shown by the various “X” symbols on FIG. 13 indicating points in the photosensitization of Riboflavin that may be inhibited by quenching agents.

Studies have shown that in the presence of light, Riboflavin can exhibit photosensitizing properties reacting with a wide range of electron donating substances (e.g., amines or amino acids) through a combination of Type I and Type II photochemical mechanisms. In the Type I mechanism, which is favored at low oxygen concentrations, excited Riboflavin reacts with compounds from the surrounding substrate to generate free radicals or radical ions by hydrogen atom or electron transfer. In the Type II mechanism, excited Riboflavin reacts with oxygen to form singlet molecular oxygen. Because Type II photosensitization requires molecular oxygen, as the oxygen in a system decreases, Type II photosensitization is shifted to Type I. The radicals/radical ions and the singlet molecular oxygen from Type I and/or Type mechanisms results in cross-linking activity of fibril structures in the corneal tissue.

A model of the photochemical kinetics of corneal cross-linking with Riboflavin in ex vivo porcine cornea was developed by the inventors. During experiments to test the model, measurements of oxygen validated the model. De-epithelialized porcine corneas were soaked with 0.1% Riboflavin in distilled water for 20 minutes and exposed to 365 nm UV light (UVA) for 30 minutes at an irradiance of 3 mW/cm² for a total dose of 5.4 J/cm². Oxygen concentrations in the cornea at a known depth in the corneal tissue were monitored during UVA illumination with a dissolved oxygen fiber optic microsensor. Data from the oxygen and temperature experiments were used to validate the model. Oxygen concentration in the cornea depends on the UVA irradiance and temperature and quickly decreases at the beginning of the procedure. The oxygen dynamics in the cornea during the activation of Riboflavin indicate that the Type-I mechanism is the predominant mechanism for cross-linking during the treatment. The Type-II mechanism is significant in providing cross-linking for a short period when the Riboflavin is initially illuminated.

Additionally, more than halfway through the illumination period in the study, the oxygen concentration in the cornea slowly increases where the Type-II mechanism may play a further role in cross-linking. During this phase, one expects a growing input from the singlet oxygen-mediated cross-linking together with the enhancement of secondary radical reactions that are modulated by oxygen. In general, however, the study suggests that the primary photochemical kinetics mechanism is the direct interaction between excited Riboflavin triplets and reactive groups of corneal proteins which leads to the cross-linking of the fibril proteins mainly through radical reactions. Thus, the inventors have established a new framework for the photochemical kinetics of corneal cross-linking with Riboflavin, and are further investigating the kinetics of free radical polymerization.

Aspects of the present disclosure control the distribution of the cross-linking agent according to the kinetics associated with its application. In particular, varying concentrations of the cross-linking agent may be achieved at various regions and depths of the corneal tissue to achieve a desired pattern of cross-linking activity. For example, the cross-linking agent may be applied by employing techniques and/or chemistries that enhance or slow the delivery of the cross-linking agent to various regions and depths of the corneal tissue. Some embodiments of the present disclosure accordingly provide ophthalmic drug application devices suitable for conveying the cross-linking agents, reverse osmotic substances, quenching agents, etc., to the corneal tissue in ordered steps to thereby regulate the distribution of the cross-linking agent within the corneal tissue.

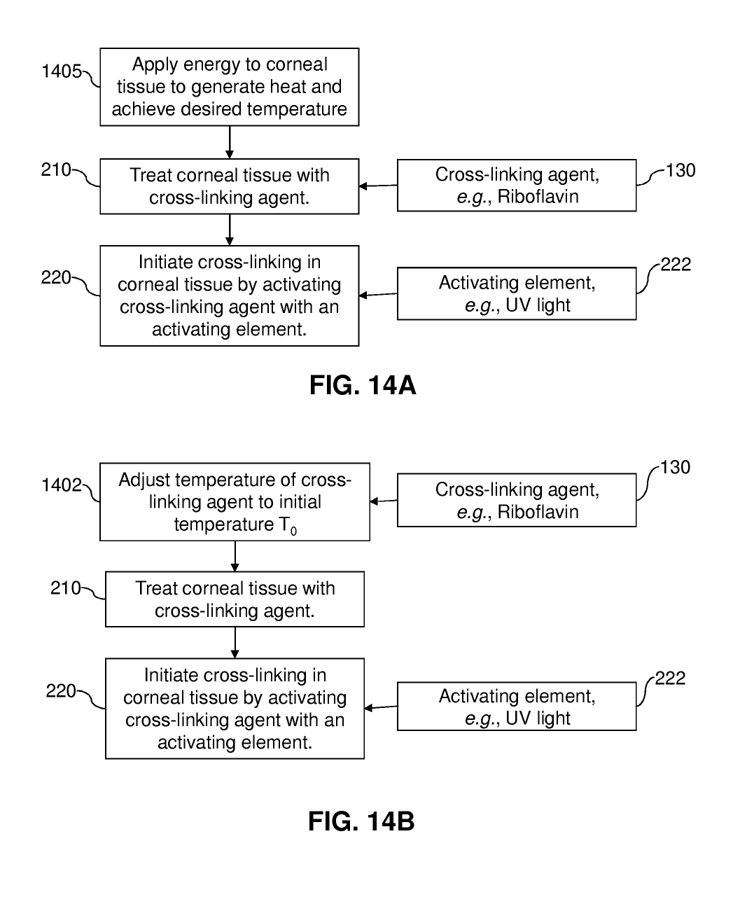

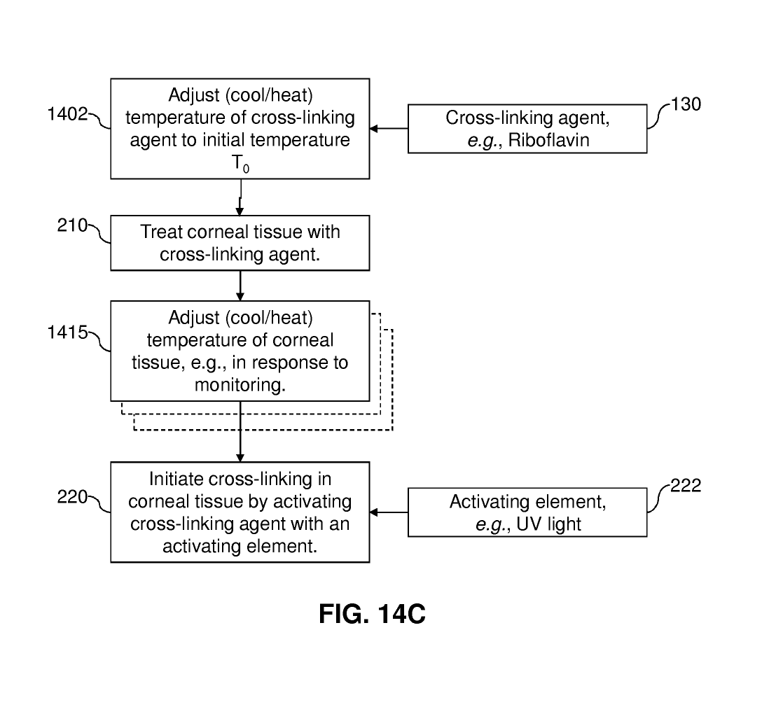

FIG. 14A illustrates another example embodiment for controlling delivery of a cross-linking agent to corneal tissue by generating heat in the corneal tissue according to aspects of the present invention. In step 1405, energy is delivered to the corneal tissue to generate heat which raises the temperature of the corneal tissue. The energy may be generated with microwaves from a device similar to the applicator described above. Alternatively, other electromagnetic energy, light energy, e.g., lasers, infrared, and/or other sources of energy, e.g., ultrasound, and the like may be applied to the corneal tissue to generate heat. Although step 1405 as shown in FIG. 19 may occur just prior to the application of cross-linking agent in step 210, the heat in the corneal tissue is generated in a manner and at a time to achieve a desired temperature for the cross-linking agent in the corneal tissue. The application of energy to increase temperature may occur contemporaneously with the cross-linking agent treatment, e.g., at any time during the soak period.

The kinetics associated with the cross-linking agent depends on temperature. In particular, diffusion by the cross-linking agent increases with temperature. For example, the diffusion coefficient of Riboflavin in corneal tissue increases by approximately a factor of 2 when the temperature of Riboflavin increases from 20° F. to 34° F. Therefore, aspects of the present invention control the distribution of a cross-linking agent in corneal tissue, at least partially, by controlling (increasing or decreasing) the temperature of the cross-linking agent in the corneal tissue.

FIG. 14B illustrates that rather than changing the temperature of the corneal tissue directly, the temperature of the cross-linking agent 130 may be adjusted (heated or cooled) in step 1402 to a desired initial temperature T₀ before it is applied in step 210. Thus, the diffusion of the cross-linking agent 130 is determined by the initial temperature T₀ when it is first applied to the corneal tissue.

FIG. 14C illustrates that the temperature of corneal tissue may also be adjusted any number of times during the cross-linking agent treatment. The cross-linking agent is applied at an initial temperature T₀ in step 1402. During the soak period, the initial temperature T₀ may be maintained at temperature T₀ or the temperature of the cross-linking agent in the eye may be adjusted in any number and combination of cooling and/or heating steps 1415 to cause the cross-linking agent 130 to move through the corneal tissue according to desired diffusion characteristics. The heating steps increase diffusion and delivery into areas/depths of corneal tissue, while the cooling steps slow down the diffusion and delivery. The temperature of the corneal tissue may be increased by applying electromagnetic energy, light energy, ultrasound, or the like, while the temperature may be decreased by applying a coolant, such as an appropriate solution at a lower temperature.

According to aspects of the present disclosure, the temperature of the cross-linking agent applied in specific areas of the cornea may be increased and/or decreased to control distribution of the cross-linking agent according to a particular pattern. As such, the distribution of the cross-linking agent throughout the corneal structure is determined at least partially by this pattern. For example, keratoconus may be treated by patterned application of a cross-linking agent, i.e., controlled delivery of the cross-linking agent throughout the corneal tissue relative to the keratoconic cone. In this treatment, the temperature of different areas of the cornea may be increased or decreased to achieve a desired distribution of the cross-linking agent relative to the keratoconic cone. The keratoconic cone may be heated or cooled directly, while the areas outside the keratoconic cone are maintained at a different temperature. Alternatively, the areas outside the keratoconic cone may be heated or cooled directly, while the keratoconic cone is maintained at a different temperature. Alternatively, the keratoconic cone may be heated or cooled directly, while the areas outside the keratoconic cone may be actively, but oppositely, cooled or heated. Such patterned heating may be achieved, for example, provided by controlled targeting of a laser.

In combination with controlling the distribution of the cross-linking agent through the use of temperature, the activation of the cross-linking agent may be controlled by patterned application of UV light, i.e., delivering specific doses of UV light to specific areas of the cornea. The patterned application of UV light determines in part the amount of cross-linking activity in the specific areas of the cornea. Systems and methods relating to controlling the application of UV light is described, for example, in U.S. patent application Ser. No. 13/051,699. For example, in the treatment of keratoconus, the keratoconic cone may be selectively illuminated with doses of UVA while the keratoconic cone is also heated. Other combinations of patterned delivery and patterned temperature control are contemplated.

FIG. 15 is a block diagram of an exemplary system 1500 for controlling the distribution of the cross-linking agent 130 within the eye 1 through permeability regulation system(s) 1520, temperature regulation system(s) 1530, and/or diffusion influencing compounds 1540 according to feedback information from feedback system(s) 1510. The system 1500 includes a controller 120, optical elements 112, and a drug application device 132, similar to the system 100 described in connection with FIG. 1. In the system 1500, the controller 120 is configured to control the permeability regulation system(s) 1520, the temperature regulation system(s) 1530, and/or the operation of the drug application device 132 to provide a desired three dimensional distribution of the cross-linking agent 130 within the corneal tissue of the eye 1. For example, the controller 120 can operate the system 1500 according to any of the methods provided by the embodiments described above. Additionally or alternatively, the system 1500 can be operated to employ at least some techniques from some embodiments and other techniques from other embodiments, such as the exemplary embodiments 1000A and 1000B of FIGS. 10A and 10B that employ certain features to achieve a desired distribution of cross-linking agent.

In an exemplary operation of the system 1500, he permeability regulation system(s) 1520 can include an ultrasound generator 1522 or a laser system 1524, or a microwave system 1526. In some examples, the controller 120 can operate to apply ultrasound energy to the corneal tissue 2 via the ultrasound generator 1522 to increase the uptake of the cross-linking agent 130 in the regions of the corneal tissue 2 receiving ultrasound energy, as described in connection with FIGS. 3-4. In some examples, the controller 120 can operate to apply heat energy to the corneal tissue 2 via the microwave system 1526 and/or laser system 1524 to increase the uptake of the cross-linking agent 130 in the regions of the corneal tissue 2 receiving heat energy, as described in connection with FIG. 14C. Thus, the controller 120 operates the permeability regulation system(s) 1520 to control the permeability (e.g., uptake rate and/or amount) of the corneal tissue 2 to the cross-linking agent 130. By applying energy from the permeability regulation system(s) 1520 according to a non-uniform pattern, the permeability of the corneal tissue 2 can be adjusted with some regions becoming relatively more permeable to the cross-linking agent than other regions, which regions are based on the applied non-uniform pattern. Modifying the corneal permeability according to a non-uniform pattern allows the cross-linking agent 130 that diffuses into the corneal tissue 2 to be non-uniform according to a pattern corresponding to the non-uniform pattern of the permeability regulation system(s) 1520.

The system 1500 further includes the feedback system(s) 1510. The feedback system(s) 1510 are configured to dynamically monitor cross-linking activity in the corneal tissue 2, the distribution of the cross-linking agent 130 within the corneal tissue 2. The feedback system(s) 1510 monitors observable factors influencing (or indicative of) the distribution of cross-linking agent or the progress of cross-linking activity within the corneal tissue 2. Generally, the feedback system(s) 1510 can include sensors to measure characteristics of the corneal tissue 2, and outputs to convey signals indicative of the measured characteristics to the controller 120. The controller 120 can be configured to adjust a cross-linking therapy and/or distribution of the cross-linking agent 130 within the eye via the optical elements 112, the drug application device 132, the permeability regulation system(s) 1520, and/or the temperature regulation systems(s) 1530.

In some examples, the feedback system(s) 1510 include an interferometry system, a multi-camera Scheimpflug system, an Ocular Coherence Tomography (OCT) system, a Supersonic Shear Imaging (SSI) system, or another monitoring system for characterizing biomechanical properties of the eye 1. For example, an interferometry system can characterize the corneal topography by comparing interference patterns of light reflected from the corneal surface with light reflected from a reference surface. Observing the corneal topography over time allows for characterization of the dynamic deformation of the corneal tissue 2 in response to subtle perturbations, such as changes in intraocular pressure, external stimuli, etc. The rate and/or amount of deformation provide an indication of biomechanical strength or stiffness (e.g., a measure of the resistance to deformation) of the corneal tissue. Thus, feedback systems 1510 operative to provide indications of the biomechanical strength of the corneal tissue 2 can indicate the progress of cross-linking activity in the eye, and thus indicate the need for additional cross-linking activity. The feedback system 1510 can optionally include systems for detecting additional biomechanical properties of the eye 1, such as corneal thickness. Further the feedback system 1510 may include a video system for monitoring the position of the cornea 2 and aligning the optical elements 112 according to the position information.

The feedback system 1510 can alternatively or additionally include an OCT system or Scheimplflug system configured to dynamically characterize the deformation response of corneal tissue 2 to subtle perturbations and thereby determine the biomechanical strength of the corneal tissue. Systems and methods relating to monitoring the distribution of cross-linking agent and aspects of cross-linking activity are described, for example, in U.S. patent application Ser. No. 13/051,699; U.S. Provisional Patent Application No. 61/492,553, filed Jun. 2, 2011; U.S. Provisional Patent Application No. 61/542,269, filed Oct. 2, 2011; U.S. Provisional Patent Application No. 61/550,576, filed Oct. 24, 2011; and U.S. Provisional Patent Application No. 61/597,137, filed Feb. 9, 2012, the contents of these applications being incorporated entirely herein by reference.

In other examples, the feedback system 1510 can include a temperature sensor to indicate the temperature of the corneal tissue 2. The temperature information from the temperature sensor can then be used to determine an amount and/or pattern of heat energy to apply to the corneal tissue 2 via, for example, the laser system 1524, an infrared light delivery system, or the microwave system 1526 of the permeability regulation system(s) 1520. As described in connection with FIG. 14C, the permeability of the corneal tissue 2 is influenced by its temperature, and so temperature information from the feedback system(s) 1510 provides indications of the diffusion characteristics of the corneal tissue 2. Based on the feedback information (e.g., temperature, etc.), applications of the cross-linking agent 130 can be adjusted with respect to concentration, diffusion/soak time, etc. to achieve a desired distribution of the cross-linking agent, similar to the discussion of iterative applications in 9A-10B. For example, diffusion of the cross-linking agent 130 may be increased with temperature if the monitoring shows that the cross-linking agent has not been delivered to particular areas or depths of the corneal tissue 2.

In some embodiments, a laser system, such as, for example, an excimer laser, can be employed to thin selected regions of the corneal tissue so as to influence the diffusion rate of cross-linking agent into the corneal tissue. For example, a laser can selectively thin the outer epithelium of the eye in regions where increased cross-linking agent uptake is desired. In some examples, the regions that are selectively thinned or otherwise altered via the laser system are determined according to feedback information indicative of the biomechanical shape and/or structure of the corneal tissue.

Additionally or alternatively, diffusion influencing compounds 1540 can be applied to the cornea 2 via the drug application device 132 to urge the cross-linking agent 130 to further depths within the cornea 2 (e.g., a neutral compound as described in connection with FIG. 8) or to draw the cross-linking agent 130 from the cornea 2 (e.g., a reverse osmotic fluid as described in connection with FIG. 7). Furthermore, cross-linking agent 130 within the corneal tissue 2 can be quenched following a cross-linking treatment by a quenching agent applied via the drug application device 132, as described in connection with FIG. 11.

In some embodiments, feedback information from the feedback system(s) 1510 can then be used to develop a treatment plan or dynamically adjust a treatment plan that is suited to the monitored characteristics of the corneal tissue 2. The treatment plan can be characterized by one or more applications of the cross-linking agent 130 to achieve desired distributions within the cornea 2 and one or more energy doses of the initiating element delivered via the optical elements 112 according to desired patterns to controllably activate cross-linking in the corneal tissue 2. Exemplary systems and methods for controlling the activation of the cross-linking agent 130 by precisely delivering the initiating element both spatially and temporally, and optionally according to information received from a feedback system are provided in U.S. patent Ser. No. 13/051,699, filed Mar. 18, 2011, and which claims priority to U.S. Provisional Application No. 61/315,840, filed Mar. 19, 2010; U.S. Provisional Application No. 61/319,111, filed Mar. 30, 2010; U.S. Provisional Application No. 61/326,527, filed Apr. 21, 2010; U.S. Provisional Application No. 61/328,138, filed Apr. 26, 2010; U.S. Provisional Application No. 61/377,024, filed Aug. 25, 2010; U.S. Provisional Application No. 61/388,963, filed Oct. 1, 2010; U.S. Provisional Application No. 61/409,103, filed Nov. 1, 2010; and U.S. Provisional Application No. 61/423,375, filed Dec. 15, 2010, the contents of these applications being incorporated entirely herein by reference. These and other techniques may be combined with the permeability regulation system(s) 1520 and/or diffusion influencing compounds 1540 to control the diffusion of the cross-linking agent 130 into selected corneal regions according to a desired distribution and thereby generate cross-linking activity at selected regions of the corneal tissue.

The embodiments above may be described with respect to treatment of the cornea and the application of a cross-linking agent. In general, however, embodiments according to the present disclosure take advantage of the increase in the permeability of eye tissue caused by the application of energy. It is further understood that aspects of the present disclosure may be employed with other eye features, such as the limbus, sclera, and retina. Thus, for example, the increased permeability may be advantageous in treating retinal membrane problems. In addition, the application of energy may be employed to produce changes in permeability in the structure of the limbus and the sclera. For example, energy may be applied to form a 6 mm to 8 mm arc at the limbus overlying Schlemm's canal. The corresponding structural changes may result in lowering intraocular pressure, for example, in the treatment of glaucoma. Increased scleral permeability without the induction of fibroblastic elements may be an effective way to lower intraocular pressure. With the increased fluid movement after energy application, significant lowering of intraocular pressure may occur. Advantageously, the amount of energy may be applied with precision.

Aspects of the present disclosure provide for lowering intraocular pressure by generating structural changes in at least one eye component of an eye. The at least one eye component may be a cornea, a limbus, a sclera, and/or a retina. According to aspects providing treatment methods for glaucoma, the cross-linking agent is not necessarily conveyed to the cornea. Aspects provide for treating glaucoma or other eye conditions by decreasing intraocular pressure with or without cross-linking also taking place. Conventional glaucoma treatments provide for decreasing intraocular pressure by regulating the flow of aqueous humor through use of, for example, prescription medications or surgical interventions. Conventional treatments to decrease intraocular pressure may decrease capillary size, or surgically redirect a flow of aqueous humor. However, aspects of the present disclosure provide for decreasing intraocular pressure to treat glaucoma by generating a structural change of an eye component by application of energy to the eye component. For example, heat energy can be applied to the eye component using the applicator 310 shown in FIG. 3. The application of heat energy generates structural changes in the corneal fibrils, which structural changes relieve intraocular pressure in the eye.

The use of Riboflavin as the cross-linking agent and UV light as the initiating element in the embodiments above is described for illustrative purposes only. In general, other types of cross-linking agents may be alternatively or additionally employed according to aspects of the present disclosure. Thus, for example Rose Bengal (4,5,6,7-tetrachloro-2′,4′,5′,7′-tetraiodofluorescein) may be employed as the cross-linking agent 130, or as the cross-linking agent delivered in varying concentrations 912, 1022. Rose Bengal has been approved for application to the eye as a stain to identify damage to conjunctival and corneal cells. However, Rose Bengal can also initiate cross-linking activity within corneal collagen to stabilize the corneal tissue and improve its biomechanical strength. Like Riboflavin, photoactivating light may be applied to initiate cross-linking activity by causing the Rose Bengal to convert O₂ in the corneal tissue into singlet oxygen. The photoactivating light may include, for example, UV light or green light. The photoactivating light may include photons having energy levels sufficient to individually convert O₂ into singlet oxygen, or may include photons having energy levels sufficient to convert O₂ into singlet oxygen in combination with other photons, or any combination thereof.

Although embodiments of the present disclosure may describe stabilizing corneal structure after treatments, such as LASIK surgery, it is understood that aspects of the present disclosure are applicable in any context where it is advantageous to form a stable three-dimensional structure of corneal tissue through cross-linking. Furthermore, while aspects of the present disclosure are described in connection with the re-shaping and/or strengthening of corneal tissue via cross-linking the corneal collagen fibrils, it is specifically noted that the present disclosure is not limited to cross-linking corneal tissue, or even cross-linking of tissue. Aspects of the present disclosure apply generally to the controlled cross-linking of fibrous matter and optionally according to feedback information. The fibrous matter can be collagen fibrils such as found in tissue or can be another organic or inorganic material that is arranged, microscopically, as a plurality of fibrils with the ability to be reshaped by generating cross-links between the fibrils. Similarly, the present disclosure is not limited to a particular type of cross-linking agent or initiating element, and it is understood that suitable cross-linking agents and initiating elements can be selected according to the particular fibrous material being reshaped and/or strengthened by cross-linking.

The present disclosure includes systems having controllers for providing various functionality to process information and determine results based on inputs. Generally, the controllers (such as the controllers 120, 340 described throughout the present disclosure) may be implemented as a combination of hardware and software elements. The hardware aspects may include combinations of operatively coupled hardware components including microprocessors, logical circuitry, communication/networking ports, digital filters, memory, or logical circuitry. The controller may be adapted to perform operations specified by a computer-executable code, which may be stored on a computer readable medium.

As described above, the controllers may be a programmable processing device, such as an external conventional computer or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGAs), digital signal processors (DSPs), micro-controllers, and the like, programmed according to the teachings of the exemplary embodiments of the present disclosure, as is appreciated by those skilled in the computer and software arts. The physical processors and/or machines may be externally networked with image capture device(s), or may be integrated to reside within an image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present disclosure for performing all or a portion (if processing is distributed) of the processing performed in implementations. Computer code devices of the exemplary embodiments of the present disclosure can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present disclosure can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the present disclosure has been described in connection with a number of exemplary embodiments, and implementations, the present disclosure is not so limited, but rather covers various modifications, and equivalent arrangements.

We claim:

1. A kit for transepithelial delivery of a cross-linking agent to a cornea, comprising:
a first vessel containing an initial formulation that includes a cross-linking agent and a first active ingredient configured to open corneal epithelial tight junctions when applied to a cornea; and
one or more second vessels each containing a respective secondary formulation that includes the cross-linking agent for application to the cornea after the initial formulation is applied to the cornea.

2. The kit of claim 1, wherein the first vessel and the one or more second vessels are syringes.

3. The kit of claim 1, further comprising another vessel containing a final formulation that includes the cross-linking agent and a second active ingredient configured to close the corneal epithelial tight junctions when applied to the cornea after the initial formulation and the one or more secondary formulations have been applied to the cornea.

4. The kit of claim 3, wherein the second active ingredient is sodium bicarbonate.

5. The kit of claim 1, wherein the one or more active formulations omit the first active ingredient.

6. The kit of claim 1, wherein the first secondary ingredient is benzalkonium chloride (BAC).

7. The kit of claim 1, further comprising another vessel containing a reverse osmotic substance.

8. The kit of claim 7, wherein the reverse osmotic substance is a gel, a hydrogel, or a semisolid substance.

9. The kit of claim 7, wherein the reverse osmotic substance is a liquid.

10. The kit of claim 9, wherein the reverse osmotic substance includes distilled water.

11. The kit of claim 1, further comprising another vessel containing a quenching substance.

12. The kit of claim 11, wherein the quenching substance includes ascorbic acid, phenolic compounds, or metal ions.

13. The kit of claim 1, wherein the cross-linking agent is Riboflavin.

14. The kit of claim 1, wherein the cross-linking agent includes a plurality of potential cross-linking agents, the secondary formulation contained in at least two of the one or more second vessels including a different one of the plurality of potential cross-linking agents.

15. A method of treating an eye, comprising:
applying an initial formulation including a cross-linking agent and a first active ingredient to a cornea to simultaneously open corneal epithelial tight junctions of the cornea and diffuse the cross-linking agent into the cornea;
after applying the initial formulation, applying one or more secondary formulations including the cross-linking agent to the cornea to further diffuse the cross-linking agent into the cornea via the open corneal epithelial tight junctions, the application of the initial formulation and the one or more secondary formulations determining a concentration profile of the cross-linking agent in the cornea; and
activating the cross-linking agent to initiate cross-linking in the cornea according to the concentration profile.

16. The method of claim 15, wherein the first active ingredient is benzalkonium chloride (BAC).

17. The method of claim 15, further comprising, after the applying the one or more secondary formulations, applying a final formulation including the cross-linking agent and a second active ingredient to the cornea to close the corneal epithelial tight junctions of the cornea, the concentration profile being determined by the application of the initial formulation, the one or more intermediate formulations, and the final formulation.

18. The method of claim 17, wherein the second active ingredient is sodium bicarbonate.

19. The method of claim 15, wherein the one or more secondary formulations omit the first active ingredient.

20. The method of claim 15, further comprising applying a reverse osmotic substance to the cornea to draw the cross-linking agent from the cornea.

21. The method of claim 20, wherein the reverse osmotic substance is a gel, a hydrogel, or a semisolid substance.

22. The method of claim 20, wherein the reverse osmotic substance is a liquid.

23. The method of claim 22, wherein the reverse osmotic substance includes distilled water.

24. The method of claim 15, further comprising:
after applying the initial formulation, waiting a first amount of time to allow the initial formulation to diffuse into the cornea;
after waiting the first amount of time, rinsing the cornea;
after each application of the one or more secondary formulations, waiting a respective second amount of time to allow each of the one or more secondary formulations to diffuse into the cornea, the concentration profile being a predetermined concentration profile, the first amount of time and each second amount of time being selected to achieve the predetermined concentration profile; and
rinsing the cornea after waiting each second amount of time.

25. The method of claim 15, wherein the activating the cross-linking includes applying ultraviolet light to the cornea.

26. The method of claim 15, wherein the cross-linking agent includes a plurality of potential cross-linking agents, at least two of the one or more secondary formulations including a different one of the plurality of potential cross-linking agents.

27. The method of claim 15, wherein the cross-linking agent includes Riboflavin.

28. The method of claim 15, wherein the cross-linking agent is activated after all of the one or more secondary formulations are applied.

29. The method of claim 28, wherein the cross-linking agent is also activated before all of the one or more secondary formulations are applied.

30. The method of claim 15, further comprising applying a quenching substance to the cornea to minimize photoactivation and unwanted cross-linking.

31. The method of claim 30, wherein the quenching substances includes ascorbic acid, phenolic compounds, or metal ions.
